(19) United States
(12) Patent Application Publication
(10) Pub. No.: US 2006/0275288 A1
(43) Pub. Date: Dec. 7, 2006

(54) GLP-1 receptor agonist and allosteric modulator monoclonal antibodies and uses thereof

(76) Inventors: Nelson D. Grihalde, Grayslake, IL (US); Christine A. Collins, Skokie, IL (US); Andrea U. Pellacani, Philadelphia, PA (US); Tariq Ghayur, Holliston, MA (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/335,387

(22) Filed: Jan. 19, 2006

Related U.S. Application Data
(60) Provisional application No. 60/645,248, filed on Jan. 20, 2005.

(51) Int. Cl. A61K 39/395; C07K 16/28

(52) U.S. Cl. 424/143.1; 530/388.22

(57) ABSTRACT

The subject invention relates to monoclonal antibodies that have the ability to bolster the function of the GLP-1 receptor and may therefore have utility in the treatment of mammalian metabolic disorders such as, for example, diabetes. In particular, the invention describes the generation of fully human monoclonal antibodies made against extracellular domains of the human GLP-1 receptor which are capable of binding the intact receptor and activating it in a manner similar to the native ligand. Additionally, the invention describes methods used to generate and develop allosteric modulator antibodies of the human GLP-1 receptor with potential therapeutic uses.

The subject application claims priority to U.S. provisional application Ser. No. 60/645,248 filed on Jan. 20, 2005 which is hereby incorporated in its entirety by reference.

### BACKGROUND OF THE INVENTION

1. Technical Field

The subject invention relates to monoclonal antibodies that have the ability to bolster the function of the GLP-1 receptor and may therefore have utility in the treatment of mammalian metabolic disorders such as, for example, diabetes. In particular, the invention describes the generation of fully human monoclonal antibodies that are made against extracellular domains of the human GLP-1 receptor and which are capable of binding the intact receptor and activating it in a manner similar to the native ligand. Further, the invention describes antibodies with allosteric modulator function that can sensitize the human GLP-1 receptor towards activation by its native ligand as well as magnify its physiological response. The invention also describes chemical modifications that may be made to antibodies of agonist or allosteric capability to conjugate peptidic or small molecule agonists to the antibodies, thereby generating much needed potent and efficacious agents for the treatment of diabetes, obesity, neurodegenerative diseases, and complications related to cardiac ischemia and reperfusion injury.

2. Background Information

A. Glucagon-like Peptide-1:

Glucagon-like peptide-1 (GLP-1) is an insulinotropic incretin that is released from L-cells in the mammalian gut (Fehman et al., eds., Frontiers in Diabetes, Vol. 13, pp. 65-229, Karger Publishing, Basel, 1997). When introduced into diabetic subjects, GLP-1 lowers plasma glucose and induces a feeling of fullness and satiety (Ehlers et al. European Association for the Study of Diabetes (EASD), 2001, Glasgow, Scotland, Poster 762). In animal models, it suppresses glucagon secretion, delays gastric emptying, lowers body weight, and promotes neogenesis of islets in the pancreas (Zander et al. EASD, 2001, Glasgow, Scotland, Poster 64; Zander et al. (2002) Lancet 359:824-830.). This spectrum of antidiabetic effects has made the GLP-1 receptor a target of numerous pharmaceutical drug discovery efforts, focused on identifying a small molecule agonist of this G-protein coupled receptor (GPCR). To date, there has been no reported success in identifying a small molecule agonist of this receptor. Effort has been focused on generating protease-resistant peptide analogs of native GLP-1 (Gotfredsen et al. EASD, 2001, Glasgow, Scotland, Poster 753; Sturis et al. (2003) British Journal of Pharmacology 140:123-132). One major limitation of the native incretin is its very short pharmacokinetic half-life (Fehman et al. (1997) supra), a combined result of renal clearance and proteolytic degradation by proteases like dipeptidylpeptidase IV (DPP IV)(Mentlein (1999) Regulatory Peptides 85:9-24). Unlike insulin (and synthetic) secretagogues exemplified by the sulfonylureas, GLP-1 receptor's insulinotropic activity is restricted to hyperglycemic environments thus rendering this incretin safer to administer than insulin. A GLP-1 receptor agonist would display this inherent safety feature, avoiding potential hypoglycemia, which is a significant adverse effect of insulin-based therapies. This would allow for a long-acting agent to be safely administered since its glucose lowering activity is limited to periods of hyperglycemia. Typical plasma half-lives of IgG antibodies can extend up to 23 days (Spiegelberg and Fishkin (1972) Clinical and Experimental Immunology 10:599-607) compared with 5 minutes for GLP-1 peptide, therefore, an antibody that can mimic the action of GLP-1 peptide will have very significant therapeutic advantages over peptide analogs of the native hormone.

B.1 Agonistic Antibodies:

Monoclonal antibodies designed to activate specific receptors have been developed (U.S. Patent Publication No. 20040091475). These were primarily generated to demonstrate feasibility of dimer-forming agents to trigger signaling of growth factor-like receptors (e.g., erythropoietin and thrombopoietin receptor). This class of growth hormone like receptors is susceptible to peptides (McConnell, et al. (1998) Journal of Biological Chemistry 379:1279-1286) or antibodies that form dimeric complexes mimicking the activity of their native ligands, which likewise promote homodimerization of two receptor subunits.

Agonistic antibodies have been described for other types of receptors including the insulin receptor and several examples of GPCRs. An agonistic monoclonal antibody that activates a mutated form of the insulin receptor associated with Rabson-Mendenhall syndrome has been described (Krook et al. (1996) Lancet 334:1582-1590).

B.2 Agonistic Antibodies that Activate GPCRs:

Historically, agonistic antibodies were discovered fortuitously when they induced physiological effects that generated a clinical pathology in conjunction with an autoimmune disease. A classic case of agonistic antibody-induced condition is Graves' disease in which autoantibodies activate the TSH receptor (Reed-Larsen et al., Williams Textbook of Endocrinology, pp. 421-422, WB Saunders Co., Philadelphia, Pa., 1998; Reed-Larsen et al. (1998) supra, p. 433).

The TSH receptor is a 744 amino acid GPCR encoded on a single protein that can be proteolytically cleaved into a 50 kDa amino-terminal region and a seven transmembrane domain region (Libert et al. (1989) Biochemical and Biophysical Research Communications (1989) 165:1250-1255). This GPCR signals through a cyclic AMP-mediated cascade. The first evidence that agonistic antibodies were responsible for triggering the TSH receptor was described by Adams and Purves in the mid 1950s (Adams (1958) Journal of Clinical Endocrinology and Metabolism 18:699-712; Adams et al. (1956) Proceedings of the University of Ontago Medical School 34:11-12). In the early days of characterizing this syndrome, it was discovered that the thyroid gland was no longer under the control of pituitary-released thyroid stimulating hormone. It became clear that circulating agonistic antibodies were continuously activating the receptor. Infusion of plasma containing these agonistic antibodies resulted in increased thyroid stimulation (Adams et al. (1974) Journal of Clinical Endocrinology and Metabolism 39:826-832). Studies conducted in gestational mothers with high agonistic antibody titers showed that their fetuses displayed high thyroid activity in response to transplacental penetration of these antibodies (Zakarija (1983) Journal of Clinical Endocrinology and Metabolism 57:1036-1040). A survey of thyroid patients suffering from Graves' disease shows that 80-100% of this population has agonist antibodies in their plasma. One observation about these patients is they do not show desensitization against the antibody even with prolonged exposure, otherwise the disease would be self-arresting (Reed-Larsen et al. supra, p. 433).

Recently, both essential hypertension and preeclampsia were found to correlate with the presence of agonistic antibodies against two distinct GPCRs. It was discovered that 44% of a cohort of hypertension patients had antibodies that reacted against the human α₁ adrenergic receptor (Hans-Peter et al. (1997) Hypertension 29:678-682). These antibodies were able to increase the beating rate of neonatal rat cardiomyocytes, suggesting they have agonistic action on the α_{1-A} adrenergic receptor. A specific inhibitor to α_{1-A} adrenergic receptor (W4101) was able to significantly attenuate this effect while an inhibitor of α_{1-B} receptor, CEC, had no influence on the chronotropic effect of these autoantibodies, suggesting that these antibodies act through the α_{1-A} receptor and not α_{1-B} receptor. A separate study showed preeclamptic gestational women (Wallukat et al. (1999) Journal of Clinical Investigation 103:945-952) possess antibodies that induce a positive chronotropic effect on neonatal rat cardiomyocytes. A synthetic peptide that corresponded to the second extracellular loop of angiotensin I (AT₁) receptor was able to attenuate this chronotropic event, suggesting that the antibody specifically targets the second extracellular loop of the AT₁ receptor. A monoclonal antibody made against the trypanosome P2 β protein cross-reacts with human β₁ adrenergic receptor in a fortuitous example of molecular mimicry (Mahler et al. (2001) European Journal of Immunology 31:2210-2216). This antibody is capable of binding to the second extracellular loop of this GPCR and inducing a positive chronotropic effect on neonatal rat cardiomyocytes. Similarly, mice immunized with the P2 β antigen experience increased heart rate, congruent with later findings that these mice have antibodies that recognized the β₁ adrenergic receptor (Sepulveda et al. (2000) Infectious Immunology 68:5114-5119).

Recently, efforts to derive an agonist monoclonal antibody against the TSH receptor have been successful (Costagliola et al. (2002) Biochemical and Biophysical Research Communications 299: 891-896; Sanders et al. (1999) Journal of Clinical Endocrinology and Metabolism 84:3797-3802; Ando et al. (2002) Journal of Clinical Investigation 110:1667-1674). These examples suggest that it is possible to generate agonistic antibodies against GPCRs that display in vivo activity. In particular, it is possible to generate a monoclonal antibody through genetic engineering, molecular evolution or chemical modification techniques that can recognize, bind and activate the human GLP-1 receptor.

B3. Allosteric Modulatory Antibodies

G protein-coupled receptors (GPCRs) represent the largest receptor superfamily in the human genome and constitute the most promising targets for drug intervention. Classic agonist and antagonist ligands designed to influence GPCRs were historically designed to target the receptor's orthosteric site, that is, the site recognized by the endogenous agonist for that receptor. Recent efforts focused on developing drugs with receptor-modulating activity have revealed that GPCRs possess additional, extracellular, allosteric binding sites that can be recognized by a variety of small molecule modulator ligands. “Allosteric sites” refer to receptor binding locations that are discrete from the ‘primary’ substrate or (orthosteric) ligand-binding site (Soudijn et al. (2001) Expert Opinion on Therapeutic Patents 11:1889-1904; see also Christopoulos A (2002) Nature Reviews Drug Discovery 1:198-210). Allosteric modulators bind to these external sites inducing conformational changes that may radically influence GPCR function. Since the endogenous ligand retains intact functionally, the overall pharmacology can resemble native contextual physiology more closely than by using constitutive synthetic agonists. Compare, for example, the insulinotropic action of sulfonylureas to allosteric modulators of the GLP-1 receptor. Sulfonylureas are prescribed as oral antidiabetic agents which act as constitutive secretagogues of insulin. Insulin secretion is induced independent of the glycemic state of the patient occasionally leading to hypoglycemic shock and other dangerous complications. Stimulation of insulin secretion through activation of the GLP-1 receptor preserves the contextual secretion of insulin which is timed to postprandial episodes when insulin is needed for glucose disposal. Therefore, allosteric modulators offer many advantages over classic orthosteric ligands as therapeutic agents, including the potential for greater GPCR-subtype selectivity and safety as well as the retention of contextual transient activation synchronous with physiological requirements. Most GPCRs are members of three major subfamilies: class A (receptors related to rhodopsin); class B (receptors related to the secretin receptor); and class C (receptors related to the metabotropic receptors). Recent reports have identified allosteric modulators for each type of GPCR. Allosteric modulators have been identified as potential pharmacophores for muscarinic and adenosine receptors, calcitonin and metabotropic glutamate and GABA B receptors (Goodman et al. (2002) Journal of American Society of Nephrology 13:1017-1024; Nemeth et al. (1998) Proceedings of the National Academy of Science USA 95:4040-4045; Nemeth et al. (2001) The Journal of Pharmacology and Experimental Therapeutics 299:323-331; see also Gasparini et al. (2002) Current Opinion in Pharmacology 2:43-49. Theoretically, all GPCR classes may therefore be subject to allosteric modulation, opening a new starting point in the search for new chemical and biologic entities modulating GPCR activity.

Allosterism in GPCRs is subtle and of multivariant manifestation, and past, traditional screening methods have generally failed to detect many allosteric modulators. More recently, there have been a number of major advances in high throughput screening, including the advent of cell-based functional assays, which have led to the discovery of more allosteric modulator mechanisms in all three GPCR classes (Mizumura et al. (1996) Circulation Research 79:415-423; Bhattacharya et al. (1996) Journal of Molecular Pharmacology 50:104-111; see also Jarvis et al. (1999) Brain Research 840:75-83).

Allosterically active monoclonal antibodies have been described for the muscarinic M2 receptor and thrombin (Peter et al. (2004) Journal of Biological Chemistry 279:55697-55706 and Colwell et al. Biochemistry (1998) 37:15057-15065) suggesting that monoclonal antibodies may be employed as potent modulators of GPCR function in the same manner that small molecule modulators have been successfully developed as drugs.

The GLP-1 receptor has demonstrated susceptibility to allosteric modulation in the presence of L-histidine (Leech et al. (2003) Endocrinology 144:4851-4858). L-Histidine potentiates cAMP response element reporter activity in INS-1 cells and in human embryonic kidney-293 (HEK 293) cells expressing GLP-1R.

All patents and publications referred to herein are hereby incorporated in their entirety by reference.

### SUMMARY OF THE INVENTION

The present invention encompasses a monoclonal antibody which binds to at least one epitope of the glucagon-like peptide-1 receptor. This epitope is located on one or more sites of the receptor selected from the group consisting of: a) the amino-terminal domain, b) extracellular loop 1, c) extracellular loop 2, d) extracellular loop 3, e) the intracellular carboxy-terminal domain, f) one or more of the three intracellular loops, g) a transmembrane peptide that transitions from the extracellular space to the transmembrane domain and h) a peptide that elicits activation of the receptor. The monoclonal antibody of the invention activates or modulates/potentiates the receptor. More specifically, activation or modulation/potentiation results in intracellular accumulation of cyclic AMP, calcium release and/or kinase-mediated phosphorylation of target protein substrates. Further, the antibody may be humanized or fully human. Additionally, the antibody may have been modified into a scFv, a minibody, a dual-specific antibody or a single-domain antibody. Preferably, the antibody functions in a manner equivalent to the native ligand of the GLP-1 receptor or activates or potentiates the activity of the GLP-1 receptor bound to a different antibody. Slight differences in functionality (e.g., binding affinity), as compared to the native ligand, may be acceptable, however, for purposes of the present invention.

The present invention also includes a hybridoma which produces the above-described monoclonal antibody. Further, the invention also includes the monoclonal antibody produced by the hybridoma.

Additionally, the present invention encompasses a method of treating a patient with Type 1 or Type 2 diabetes mellitus. This method comprises administering a monoclonal antibody (e.g., agonistic or allosteric modulator), to the patient, which binds to the GLP-1 receptor and activates (with an agonistic antibody) or modulates (with an allosteric modulator) the receptor. The antibody is administered in an amount sufficient to effect the treatment. The antibody causes at least one of the following results: induction of insulin secretion, suppression of glucagon release, improvement of glycemic control, promotion of islet neogenesis, delay of gastric emptying or potentiation of glucose resistant islets.

Furthermore, the present invention includes a method of treating a patient with a neurodegenerative disorder, a cognitive disorder, memory disorder or learning disorder comprising administering to said patient a monoclonal antibody which binds to a GLP-1 receptor and activates or modulates the receptor, in an amount sufficient to effect treatment. The neurodegenerative disorder may be, for example, dementia, senile dementia, mild cognitive impairment, Alzheimer-related dementia, Huntington's chores, tardive dyskinesia, hyperkinesias, mania, Morbus Parkinson, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis, glaucoma and Alzheimer's disease.

The present invention also includes a method of treating a patient with a metabolic disorder selected from the group consisting of obesity, metabolic syndrome X and pathologies emanating from islet insufficiency comprising administering to said patient a monoclonal antibody which binds to a GLP-1 receptor and activates or modulates/potentiates the receptor, in an amount sufficient to effect treatment.

Moreover, the present invention also includes a method of producing a monoclonal antibody (i.e., either agonistic or allosteric modulator antibody) that binds to and activates (in the case of the agonistic antibody) or binds to and modulates (in the case of the allosteric modulator antibody) the GLP-1 receptor. This method comprises the steps of providing an antigen that comprises a structural feature of at least one receptor domain of the GLP-1 receptor; exposing an antibody library to the antigen; selecting, from the antibody library, an antibody that binds to the receptor and activates or modulates the receptor, wherein the antibody is monoclonal. The antigen may be, for example, a cyclic peptide which mimics a structural loop of the GLP-1 receptor, a hybrid peptide comprising alternating regions of the GLP-1 receptor and a non-GLP-1 peptide (e.g., PTH or TSHR) that presents one or more loops and at least one extracellular domain of the GLP-1 receptor in an antigenic manner or a hybrid peptide in which one peptide has been introduced into a full-length protein, wherein the one peptide and full-length peptide are functional regions of two different, structurally related proteins.

Further, the present invention includes another method of producing a monoclonal antibody which binds to and activates or modulates a GLP-1 receptor. This method comprises the steps of transfecting a nucleotide sequence encoding the receptor or a hybrid thereof into a cell line for a time and under conditions sufficient for the transfected cell line to express the GLP-1 receptor, injecting the cell line into a mammal, and generating a hybridoma from lymphocytes of the injected mammal, wherein the hybridoma produces the monoclonal antibody which binds to and activates or modulates the GLP-1 receptor.

Further, the present invention includes an additional method of producing a monoclonal antibody which binds to and activates or modulates the GLP-1 receptor. This additional method comprises the steps of cloning a nucleic acid molecule encoding a GLP-1 receptor or hybrid GLP-1 receptor into a vector; injecting a mammal with the vector for a time and under conditions sufficient for the mammal to produce the antigen, screening an antibody library produced by the immunized mammal for an antibody produced against the antigen, wherein the antibody binds to and activates or modulates the GLP-1 receptor and is monoclonal. The mammal may be, for example, a nonhuman mammal such as a rabbit or a chimpanzee. Also, the nonhuman mammal can be a transgenic animal with a humanized immune repertoire and as such is capable of generating human antibodies with the desired properties defined supra.

Furthermore, the present invention includes another method of producing a monoclonal antibody that binds to and activates or modulates the GLP-1 receptor. This method comprises the steps of immunizing a nonhuman mammal with a GLP-1 receptor antigen which stimulates an antibody response in the mammal; preparing and screening a phage display antibody library using immunoglobulin sequences from lymphocytes stimulated in vivo by exposure to the GLP-1 receptor antigen; and selecting an antibody from the screened phage display antibody library, wherein the selected antibody is monoclonal and binds to and activates or modulates the GLP-1 receptor.

Additionally, the present invention includes allosteric modulator antibodies which, in the presence of the cognate ligand GLP-1, enhance the sensitivity of the human GLP-1 receptor (GLP1R) as well as magnify its physiological responses. In particular, the present invention includes a monoclonal antibody which binds to the GLP-1 receptor and potentiates its function in the presence of an endogenous or exogenous agonist. The present invention also includes a chemically modified antibody comprising at least one synthetic agonist peptide coupled to the allosteric modulator antibody.

Moreover, the present invention also encompasses a pharmaceutical composition comprising: a) a monoclonal antibody that binds to and activates the GLP-1 receptor and b) a pharmaceutically acceptable carrier.

Further, the present invention includes a pharmaceutical composition comprising: a) a monoclonal antibody that 1) binds to and 2) activates or potentiates the GLP-1 receptor; b) a pharmaceutical dose of a composition selected from the group consisting of a DPP IV inhibitor, a NEP inhibitor, a GLP-1 secretagogue, a GIP secretagogue and a protease resistant agonist of GLP1R; and c) a pharmaceutically acceptable carrier.

Additionally, the present invention encompasses a monoclonal antibody having at least one characteristic selected from the group consisting of: a) improving sensitivity of the GLP-1 receptor such that physiological activation of the receptor is achieved in the presence of an endogenous or exogenous agonist; b) magnifying the response of the receptor such that resultant in vitro intracellular cyclic AMP response to the agonist is increased as compared to response with agonist alone; and c) improving an in vivo response selected from the group consisting of: 1) glycemic control, 2) reduction of production of glycated hemoglobin, 3) reduction of glucagon secretion, 4) glucose sensitivity and 5) preservation of islet structure and function.

Further, the present invention includes a monoclonal antibody (5A10) produced by a hybridoma designated by American Type Culture Collection deposit number ______, as well as the hybridoma which produces this monoclonal antibody.

Also, the present invention encompasses a monoclonal antibody (9A10) produced by a hybridoma designated by American Type Culture Collection deposit number ______, as well the hybridoma that produces the monoclonal antibody.

It should also be noted that the present invention includes a chemically modified antibody comprising at least one synthetic agonist peptide coupled to the monoclonal antibody (i.e., agonistic or allosteric modulator) or antibodies described above.

Further, the present invention also encompasses a pharmaceutical composition comprising: a) a monoclonal antibody that binds to and potentiates or modulates the GLP-1 receptor and b) a pharmaceutically acceptable carrier.

### BRIEF DESCRIPTION OF THE DRAWINGS

FIG. 1 includes the nucleotide sequence of the human gene (SEQ ID NO:1) encoding the GLP-1 receptor. Further, this figure also includes the nucleotide sequence of the rat gene (SEQ ID NO:2) encoding the rat GLP-1 receptor and the nucleotide sequence of the mouse gene (SEQ ID NO:3) encoding the mouse GLP-1 receptor.

FIG. 2 includes the amino acid sequence of the human GLP-1 receptor (SEQ ID NO:4) as well as the amino acid sequence of the rat GLP-1 receptor (SEQ ID NO:5) and the mouse GLP-1 receptor (SEQ ID NO:6).

FIG. 3 represents the structure of the GLP-1 receptor. Transmembrane, extracellular and intracellular domains are illustrated.

FIG. 4 illustrates the interaction between the agonist antibody of the present invention and the GLP-1 receptor. In particular, the antibody initially binds the receptor. Subsequently, the antibody activates the receptor which forms regions of high receptor density called “coated pits”. Then, the coated pits internalize and begin intracellular signaling cascades. It should be noted, however, that the illustration represents only one theoretical mode of action. Other modes of action are possible. Further, the agonist antibody may not undergo all aspects of the activation cascade (i.e., internalization, coated pit formation and adenylate cyclase activation).

FIG. 5 illustrates a FACS analysis which shows GLP1R specific antibodies derived from whole cell immunization display selective binding for transfected cells displaying the human GLP1R.

FIG. 6 illustrates that monoclonal antibodies 5A10 and 9A10 allosterically enhance intracellular cAMP response of human GLP1R while mAb 2A9 is allosterically inert.

FIG. 7 illustrates that monoclonal antibodies 5A10 and 9A10 maintain allosteric activity between 100 and 1000 picomolar of GLP-1 in an antibody dose-dependent manner.

FIG. 8 illustrates that allosteric antibodies increase the sensitivity of GLP1R while amplifying the magnitude of cAMP response to GLP-1 peptide.

FIG. 9 shows that monoclonal antibody 9A10 retains functional activity on a CHO cell line expressing a low density (4×10³ receptors per cell) of GLP1R at a physiological concentration of GLP-1 peptide.

FIG. 10 illustrates the amino acid sequences of example allosteric monoclonal antibodies of differing isotype.

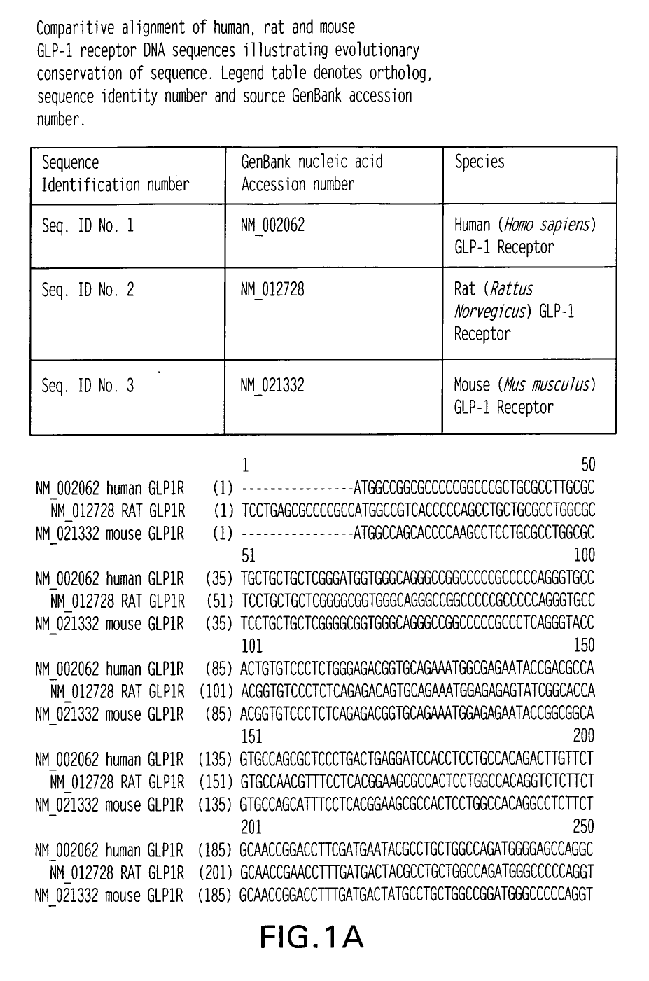

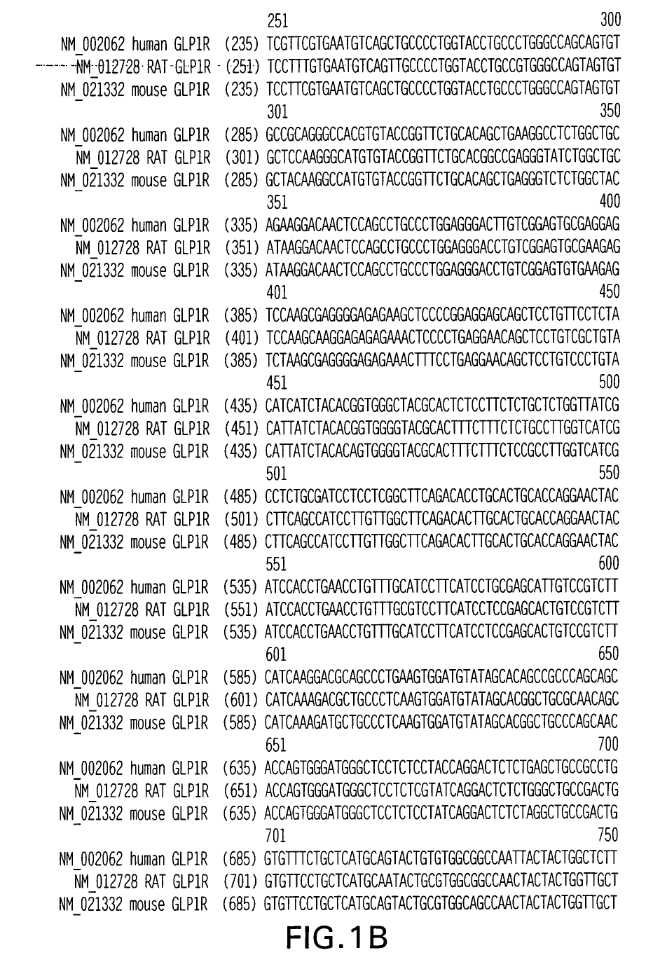

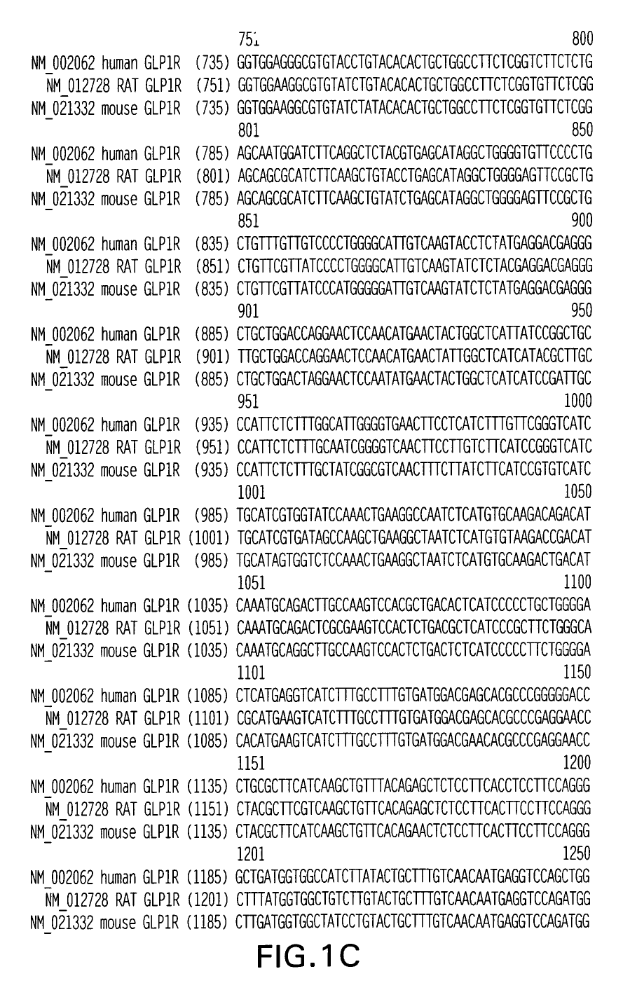

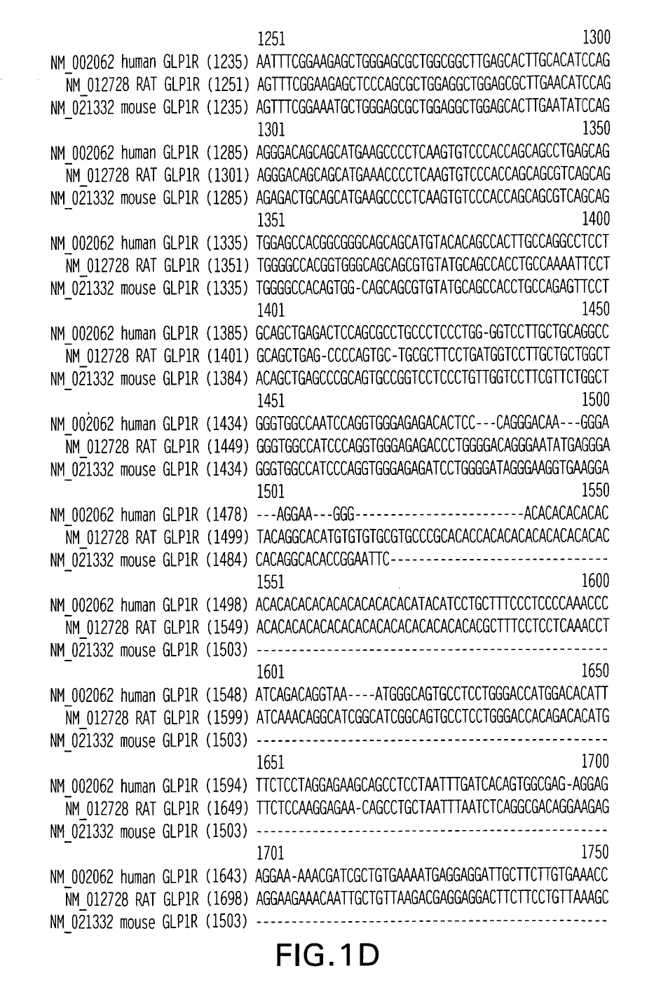

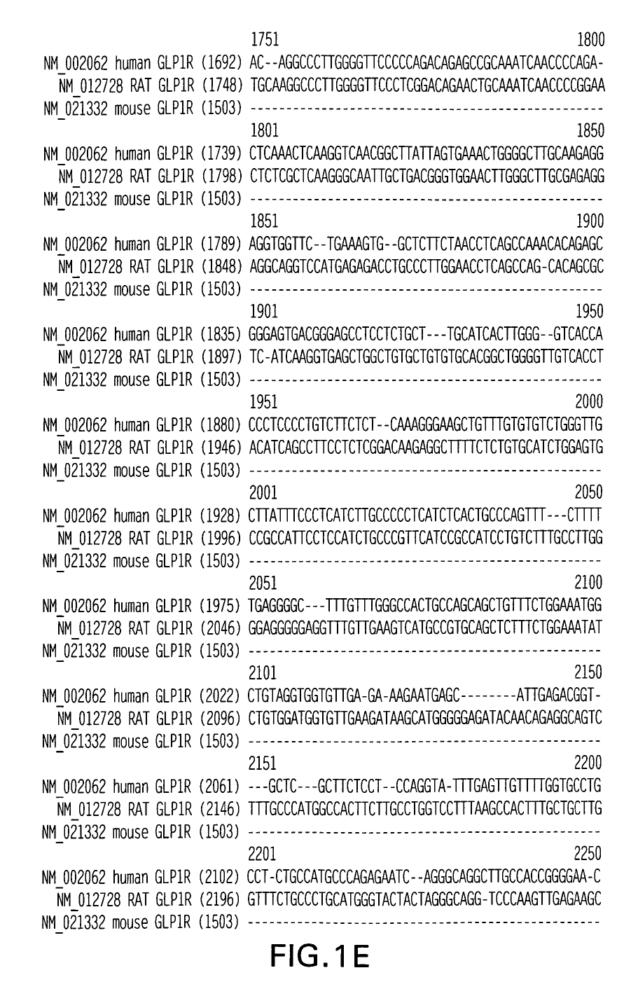

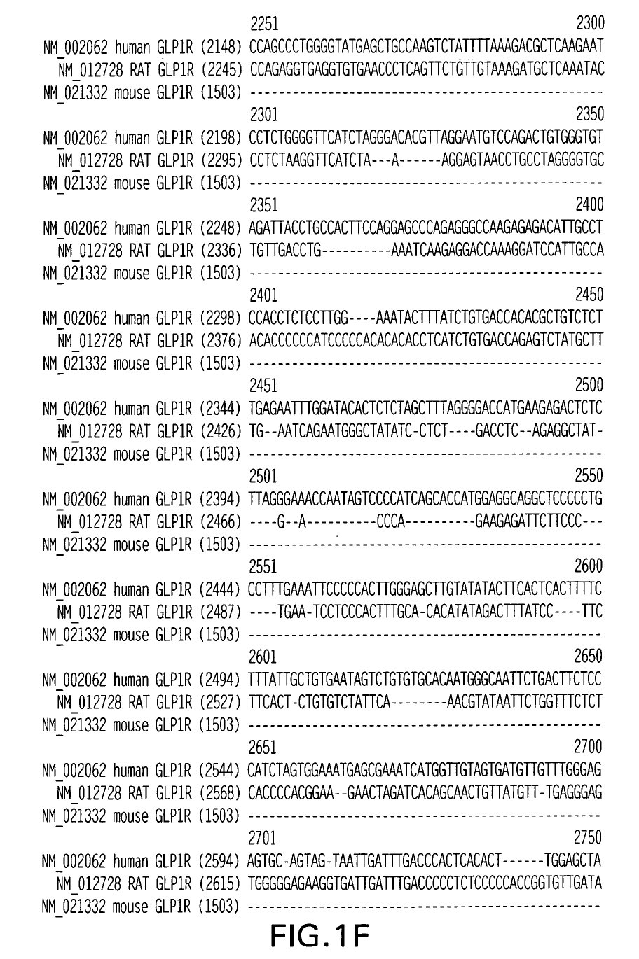

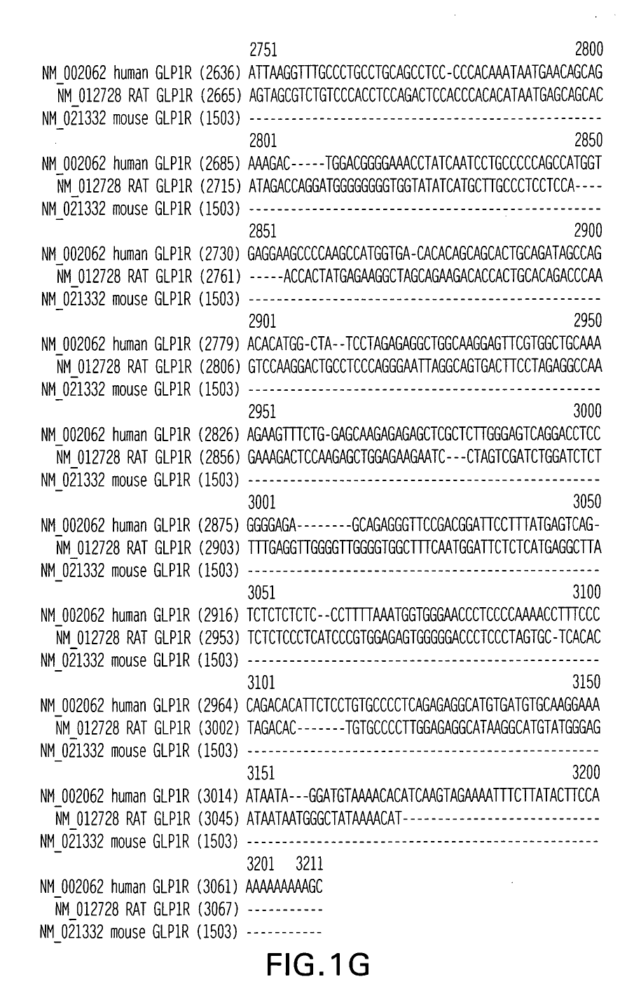

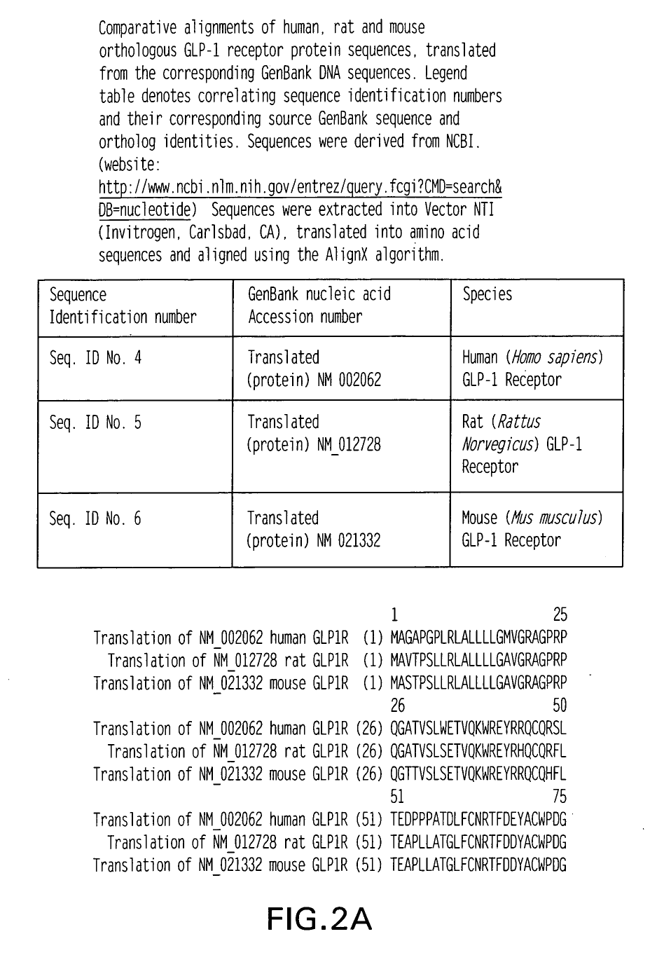

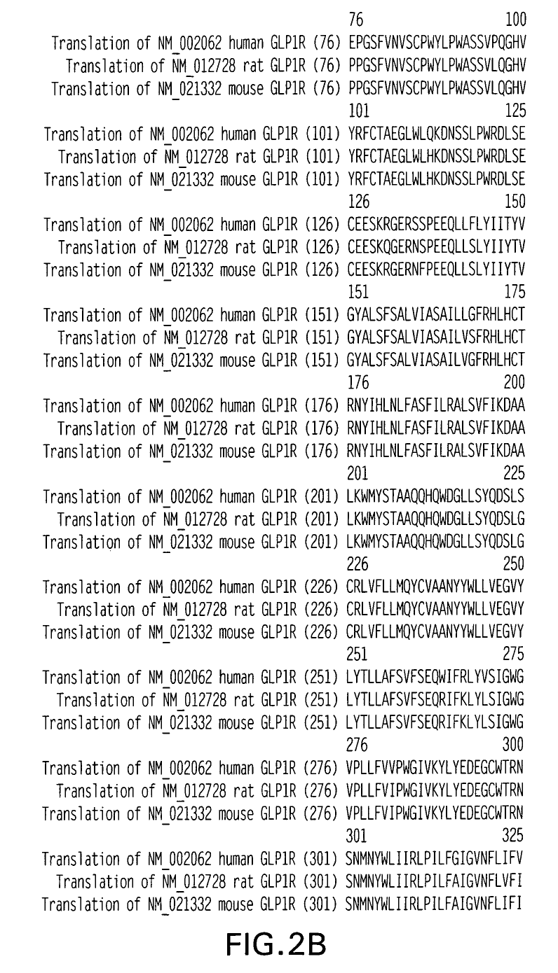

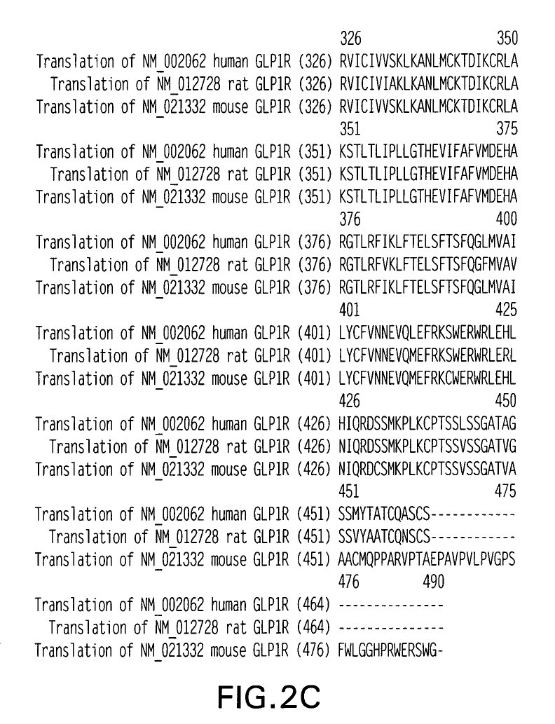

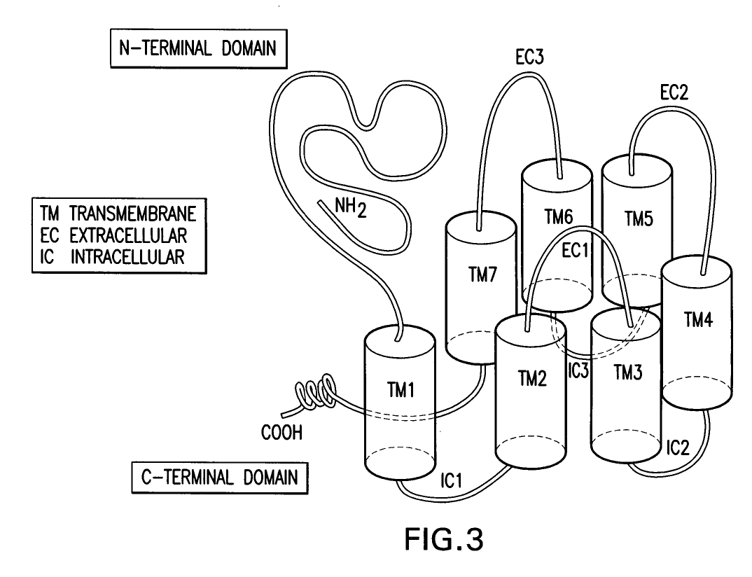

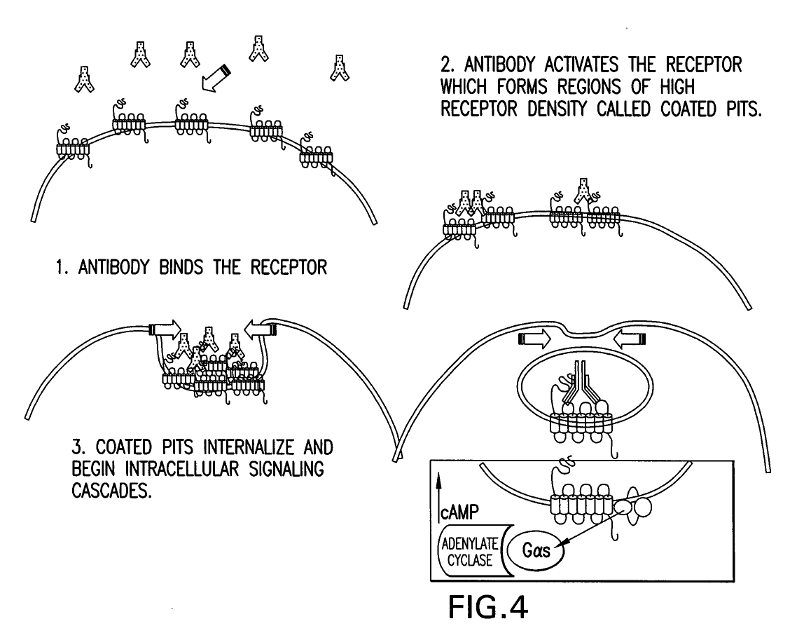

### DETAILED DESCRIPTION OF THE INVENTION

The present invention relates to the design, production and use of an agonistic antibody that can activate GPCRs and an allosteric modulator antibody that can modulate GPCRs, particularly the human GLP-1 receptor, as well as the selection, preparation and use of such antibodies. GPCRs are a subset of seven transmembrane domain receptors having common structural features that include an extracellular amino-terminal domain, and seven transmembrane lipophilic helical domains alternately connected by three extracellular and three intracellular loops and terminated by an intracellular carboxy-terminal tail. GPCRs can be divided into several classes of which GLP-1 receptor, glucagon receptor, VIP receptor and PACAP receptor belong to class II. In order to produce such an antibody that specifically binds the GLP-1 receptor and leads to activation of the receptor, one first isolates an antigen that comprises a structural feature of the receptor's extracellular domains. One then exposes an antibody repertoire to the antigen, and selects from the repertoire an antibody that specifically binds the receptor and activates or modulates the receptor. This method is just one example of many methods that may be used to produce the antibodies of the present invention, as will be described in detail below.

Further, it should be noted that while the antibodies of the present invention are described herein in terms of recognition of extracellular domains of the receptor, it should be understood that the term “GLP-1 receptor agonist antibody” or “GLP-1 receptor allosteric modulator antibody” is intended to include any antibody that specifically recognizes even intracellular loops, transmembrane domains or fusions of any of the presently mentioned antigens. This may include, for example, structurally related but distinct antigens that mimic the GLP-1 receptor. Thus, chimeric receptors that have portions analogous to the GLP-1 receptor fused to corresponding sections of another GPCR, such as glucagon receptor or thyroid stimulating hormone receptor, may also serve as antigens for the generation of agonist or allosteric modulator antibodies.

To prepare an agonist antibody of the invention, the antibody is raised against an antigen capable of eliciting production of the agonistic antibody or antibodies. Such antigens generally are referred to herein as “agonistic antigens”. The present invention includes the antibodies raised against the agonistic antigens, in particular, isolated agonistic antibodies, as well as antibody portions thereof, prepared in accordance with the methods of the invention. Further, the antibodies of the invention include monoclonal and recombinant antibodies, and portions thereof. In various embodiments, the antibody, or portion thereof, may comprise amino acid sequences derived entirely from a single species, such as a fully human or fully mouse antibody, or portion thereof. In other embodiments, the antibody, or portion thereof, may be a chimeric antibody or a CDR-grafted antibody (CDR, complementary determining region) or other form of humanized antibody.

The term “antibody”, as used herein, is intended to refer to immunoglobulin molecules comprised of four polypeptide chains, two heavy (H) chains and two light (L) chains interconnected by disulfide bonds. Each heavy chain is comprised of a heavy chain variable region (abbreviated herein as HCVR or VH) and a heavy chain constant region. The heavy chain constant region is comprised of three domains, CH1, CH2 and CH3 (constant region, heavy chain). Each light chain is comprised of a light chain variable region (abbreviated herein as LCVR or VL) and a light chain constant region. The light chain constant region is comprised of one domain, CL (constant region, light chain). The VH and VL regions can be further subdivided into regions of hypervariability, termed complementarity determining regions, interspersed with regions that are more conserved, termed framework regions (FR). Each VH and VL (variable regions, heavy chain and light chain, respectively) is composed of three CDRs and four FRs, arranged from amino-terminus to carboxy-terminus in the following order: FR1, CDR1, FR2, CDR2, FR3, CDR3, FR4. CDRs comprise regions of highest sequence diversity, while FRs serve as scaffolding to mechanically support and present the CDRs in a spatial arrangement that allows them to recognize and bind specific epitopes. The CDRs contribute a significant amount of binding energy to the antigen binding portion of the antibody.

The term “antigen-binding portion” of an antibody (or simply “antigen portion”), as used herein, refers to one or more fragments of an agonistic or allosteric modulator antibody that retain(s) the ability to specifically bind the receptor and activate or modulate it, respectively. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al. (1989) Nature 341:544-546), which consists of a VH domain; and (vi) an isolated CDR. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv) antibodies. (See, e.g., Bird et al. (1988) Science 242:423-426; Huston et al. (1988) Proceedings of the National Academy of Science USA 85:5879-5883.) Such scFv antibodies are also intended to be encompassed within the term antigen-binding portion of an antibody. Other forms of single chain antibodies, such as diabodies are also encompassed within the term. Diabodies are bivalent, bispecific, antibodies in which VH and VL domains are expressed on a single polypeptide chain, but using a linker that is too short to allow for pairing between the two domains on the same chain, thereby forcing the domains to pair with complementary domains of another chain and creating two antigen binding sites on the same receptor or across two receptor molecules. (See, e.g., Holliger et al. (1993) Proceedings of the National Academy of Science USA 90:6444-6448; Poljak et al. (1994) Structure 2:1121-1123.)

Still further, an antibody or antigen-binding portion thereof may be part of a larger immunoadhesion molecule, formed by covalent or non-covalent association of the antibody or antibody portion with one or more other or different proteins or peptides. Examples of such immunoadhesion molecules include use of the streptavidin core region to make a tetrameric scFv molecule (Kipriyanov et al. (1995) Human Antibodies and Hybridomas 6:93-101) and use of a cysteine residue, a marker peptide and a C-terminal polyhistidine tag to make bivalent and biotinylated scFv molecules (Kipriyanov et al. (1994) Molecular Immunology 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques.

An “isolated agonistic antibody” or “allosteric modulator antibody”, as used herein, is intended to refer to an antibody that is substantially free of other antibodies having different antigenic specificities (e.g., an isolated antibody that specifically binds an agonistic epitope on the receptor, but that is substantially free of other antibodies that specifically bind other epitopes on the same antigen). Moreover, an isolated agonistic antibody or isolated allosteric modulator antibody may be substantially free of other cellular material and/or chemicals.

An “agonistic antibody”, as used herein, is intended to refer to an antibody whose binding to a particular antigen results in activation of the biological activity of the antigen. This activation of the biological activity of the antigen can be assessed by measuring one or more indicators of biological activity of the antigen using an appropriate in vitro or in vivo assay.

A “monoclonal antibody” as used herein is intended to refer to a preparation of antibody molecules which share a common heavy chain and common light chain amino acid sequence, in contrast with “polyclonal” antibody preparations which contain a mixture of different antibodies. Monoclonal antibodies can be generated by several novel technologies like phage, bacteria, yeast or ribosomal display, as well as classical methods exemplified by hybridoma-derived antibodies (e.g., an antibody secreted by a hybridoma prepared by hybridoma technology, such as the standard Kohler and Milstein hybridoma methodology ((1975) Nature 256:495-497). Thus, a non-hybridoma-derived agonistic antibody of the invention is still referred to as a monoclonal antibody although it may have been derived by non-classical methodologies.

The phrase “recombinant antibody” refers to antibodies that are prepared, expressed, created or isolated by recombinant means, such as antibodies expressed using a recombinant expression vector transfected into a host cell, antibodies isolated from a recombinant, combinatorial antibody library, antibodies isolated from an animal (e.g., a mouse) that is transgenic for human immunoglobulin genes (see, e.g., Taylor et al. (1992) Nucleic Acids Research 20:6287-6295) or antibodies prepared, expressed, created or isolated by any other means that involves splicing of particular immunoglobulin gene sequences (such as human immunoglobulin gene sequences) to other DNA sequences. Examples of recombinant antibodies include chimeric, CDR-grafted and humanized antibodies.

The term “human antibody” refers to antibodies having variable and constant regions corresponding to, or derived from, human germline immunoglobulin sequences (e.g., see Kabat et al. Sequences of Proteins of Immunological Interest, Fifth Edition, U.S. Department of Health and Human Services, NIH Publication No. 91-3242, 1991). The human antibodies of the present invention, however, may include amino acid residues not encoded by human germline immunoglobulin sequences (e.g., mutations introduced by random or site-specific mutagenesis in vitro or by somatic mutation in vivo), for example, in the CDRs and, in particular, CDR3.

Recombinant human antibodies of the present invention have variable regions, and may also include constant regions, derived from human germline immunoglobulin sequences. (See Kabat et al. (1991) supra.) In certain embodiments, however, such recombinant human antibodies are subjected to in vitro mutagenesis (or, when an animal transgenic for human Ig sequences is used, in vivo somatic mutagenesis), and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis or backmutation or both.

The term “backmutation” refers to a process in which some or all of the somatically mutated amino acids of a human antibody are replaced with the corresponding germline residues from a homologous germline antibody sequence. The heavy and light chain sequences of a human antibody of the invention are aligned separately with the germline sequences in the VBASE database to identify the sequences with the highest homology. VBASE is a comprehensive directory of all human germline variable region sequences compiled from published sequences, including current releases of GenBank and EMBL data libraries. The database has been developed at the MRC Centre for Protein Engineering (Cambridge, UK) as a depository of the sequenced human antibody genes (website: http://www.mrc-cpe.cam.ac.uk/vbase-intro.php?menu=901). Differences in the human antibody of the invention are returned to the germline sequence by mutating defined nucleotide positions encoding such different amino acids. The role of each amino acid thus identified as a candidate for backmutation should be investigated for a direct or indirect role in antigen binding, and any amino acid found after mutation to affect any desirable characteristic of the human antibody should not be included in the final human antibody. To minimize the number of amino acids subject to backmutation, those amino acid positions found to be different from the closest germline sequence, but identical to the corresponding amino acid in a second germline sequence, can remain, provided that the second germline sequence is identical and co-linear to the sequence of the human antibody of the invention for at least 10, preferably 12, amino acids on both sides of the amino acid in question. Backmutation may occur at any stage of antibody optimization.

The term “chimeric antibody” refers to antibodies which comprise heavy and light chain variable region sequences from one species and constant region sequences from another species, such as antibodies having murine heavy and light chain variable regions linked to human constant regions.

The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “humanized antibody” refers to antibodies which comprise heavy and light chain variable region sequences from a nonhuman species (e.g., a mouse) but in which at least a portion of the VH and/or VL sequence has been altered to be more “human-like”, i.e., more similar to human germline variable sequences. One type of humanized antibody is a CDR-grafted antibody in which human CDR sequences are introduced into nonhuman VH and VL sequences to replace the corresponding nonhuman CDR sequences.

The term “epitope” is defined herein as a region of the antigen that binds to the antibody. In general, epitopes are comprised by local surface structures that can be formed by contiguous or noncontiguous amino acid sequences.

The term “immunize” refers herein to the process of presenting an agonistic antigen to an immune repertoire whether that repertoire exists in a natural genetically unaltered organism, or a transgenic organism modified to display an artificial human immune repertoire. Similarly, an “immunogenic preparation” is a formulation of antigen that contains adjuvants or other additives that would enhance the immunogenicity of the antigen. An example of this would be coinjection of a purified form of GLP-1 receptor with Freund's complete adjuvant into a mouse. “Hyperimmunization”, as defined herein, is the act of serial, multiple presentations of an antigen in an immunogenic preparation to a host animal with the intention of developing a strong immune response.

One way of measuring the binding kinetics of an antibody is by surface plasmon resonance. The term “surface plasmon resonance”, as used herein, refers to an optical phenomenon that allows for the analysis of real-time biospecific interactions by detection of alterations in protein concentrations within a biosensor matrix, for example using the Biacore system (Biacore International, Upsala, Sweden and Piscataway, N.J.). For further descriptions, see Jönsson et al. (1993) Annales de Biologie Clinique (Paris) 51:19-26; Jönsson et al. (1991) Biotechniques 11:620-627; Johnsson et al. (1995) Journal of Molecular Recognition 8:125-131; and Johnnson et al. (1991) Analytical Biochemistry 198:268-277.

The term “k_{off}”, as used herein, is intended to refer to the off rate kinetic constant for dissociation of an antibody from the antibody/antigen complex.

The term “kₒₙ” as used herein, is intended to refer to the on rate kinetic constant of an antibody to the target antigen.

The term “K_{d}”, as used herein, is intended to refer to the equilibrium dissociation constant of a particular antibody-antigen interaction. It is conventionally defined as the ratio between the kinetic off rate over the kinetic on rate or K_{d}=[k_{off}/kₒₙ].

The term “activation”, as used herein, is intended to refer to any cellular response that causes the cell to execute functions normally associated with the GLP-1 receptor when it binds its native ligand GLP-1 peptide. This includes, for example, intracellular accumulation of cyclic AMP, calcium release, and kinase mediated phophorylation of target protein substrates.

Also, the term “hybrid peptide”, as used herein, refers to a non-immunogenic GLP-1 receptor peptide which is coupled to another peptide from another protein (for example, a glucagon receptor or a foreign protein, such as green fluorescent protein) such that the non-immunogenic GLP-1 receptor peptide induces an immune response when coupled to the other protein.

The term “structurally-related proteins”, as used herein, is intended to encompass two proteins that belong to the same or related class of GCPRs having homologous and corresponding domains, for example, three extracellular loops, three intracellular loops and one amino-terminal domain and one carboxy-terminal domain. For instance, a structurally related protein may be an expressed GLP-1 extracellular loop on the glucagon receptor (i.e., a highly similar GPCR belonging to the same class of receptor).

The term “modulator” defines a molecule that is able to influence the functional activity of a receptor. The term “allosteric modulation” refers to the control of a receptor's binding site by a modulator molecule that binds to a regulatory site located outside of the cognate ligand binding site. In contrast, “orthosteric modulator” refers to a modulator molecule that directly occupies or interferes with the cognate ligand's binding site on the receptor. The term “positive allosteric modulator” refers to a modulator molecule that increases the sensitivity or the physiological response magnitude of a receptor following activation by its native ligand. Such a phenomenon can be monitored by measuring the increase in magnitude of the physiological response triggered by a receptor exemplified by cyclic AMP accumulation, calcium release, kinase activity or insulin secretion.

The term “allosteric antibody” or “allosteric modulator antibody” refers to an antibody molecule that is capable of modulating a receptor, exemplified by the GLP-1 receptor, only in the presence of its cognate ligand. The term “potentiator” refers to a modulator molecule that confers positive, allosteric modulatory activity to a receptor. A “potentiating antibody” refers to an antibody that confers positive, allosteric modulatory activity on a receptor. The term “inert” antibody refers to absence of any modulatory activity either positive or negative toward a receptor, specifically the GLP-1R.

The acronym “DPP IV” denotes the enzyme dipeptidyl peptidase IV, “NEP” refers to neutral endopeptidase and “GIP” refers to Glucose-dependent insulinotropic polypeptide. The term “incretin” refers to hormones GLP-1 and GIP.

The agonistic antibodies of the invention are prepared using any of the various methods for preparing antibodies described above. Further, the agonistic antibodies of the invention may be directed against essentially any structurally related antigens, although preferred agonistic antibodies of the invention are those that specifically bind an agonistic epitope of the GLP-1 receptor which can be prepared using an agonistic antigen such as described in Example 1. Other structurally related antigens that can be applied to the current invention include, but are not limited to, PTH parathyroid hormone receptor family members, cytokine families, such as leptin receptor, human growth factor receptor and erythropoietin receptor family members.

The binding activity of the agonistic or allosteric modulator antibodies toward the structurally related antigens, as well as toward hybrid agonistic or allosteric modulator antigens, can be assessed using standard in vitro immunoassays, such as ELISA or Biacore analysis as well as fluorescence activated cell sorting (FACS).

Preferably the EC₅₀s of agonistic or allosteric modulator antibodies for the target antigens are close to the EC₅₀ of the native ligand for the receptor in a given bioassay.

An agonistic or allosteric modulator antibody, or antigen-binding portion thereof, of the invention is preferably selected to have desirable binding kinetics (e.g., high affinity, low dissociation, slow off-rate) For example, the agonistic or allosteric modulator antibody, or portion thereof, may bind the antigen with a k_{off} rate constant of 0.1 s⁻¹ or less, more preferably a k_{off} rate constant of 1×10⁻² s⁻¹ or less, even more preferably a k_{off} rate constant of 1×10⁻³ s⁻¹ or less, even more preferably a k_{off} rate constant of 1×10⁻⁴ s⁻¹ or less, or even more preferably a k_{off} rate constant of 1×10⁻⁵ s⁻¹ or less, as determined by surface plasmon resonance. Alternatively, an agonistic or allosteric modulator antibody, or portion thereof, may activate the GLP-1 receptor with an EC₅₀ of 1×10⁻⁶ M or less, even more preferably with an EC₅₀ of 1×10⁻⁷ M or less, even more preferably with an EC50 of 1×10⁻⁸ M or less, even more preferably with an EC₅₀ of 1×10⁻⁹M or less, even more preferably with an EC₅₀ of 1×10⁻¹⁰ M or less, or even more preferably with an EC₅₀ of 1×10⁻¹¹ M or less. Preferably, EC₅₀ should be measured using a sensitive bioassay where GLP-1 receptors from mouse, rat, monkey, dog, as well as human, are evaluated.

The term “minibody” refers to a minimized antibody that comprises a VL a VH and a CH1 domain as defined supra.

The invention also provides a pharmaceutical composition comprising an agonistic or allosteric modulator antibody, or antigen-binding portion thereof, and a pharmaceutically acceptable carrier. The pharmaceutical composition of the invention can furt her comprise at least one additional therapeutic agent, e.g., one or more additional therapeutic agents for treating a disorder in which use of the agonistic or allosteric modulator antibody is beneficial to amelioration of the disorder. For example, when the agonistic or allosteric modulator antibody specifically binds the GLP-1 receptor, the pharmaceutical composition can further include one or more additional therapeutic agents for treating disorders of diabetes mellitus.

The antibodies and antibody portions of the present invention can be incorporated into pharmaceutical compositions suitable for administration to a subject. Typically, the pharmaceutical composition comprises an antibody or antibody portion of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like, that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols (such as mannitol, sorbitol), or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

The antibodies and antibody portions of the invention can be incorporated into a pharmaceutical composition suitable for parenteral administration. Preferably, the antibody or antibody portions will be prepared as an injectable solution containing 0.1-250 mg/ml antibody. The injectable solution can be composed of either a liquid or lyophilized dosage form in a flint or amber vial, ampule or pre-filled syringe. The buffer can be L-histidine (1-50 mM), optimally 5-10 mM, at pH 5.0 to 7.0 (optimally pH 6.0). Other suitable buffers include but are not limited to, sodium succinate, sodium citrate, sodium phosphate or potassium phosphate. Sodium chloride can be used to modify the tonicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-methionine (optimally 5-10 mM). Other suitable bulking agents, including glycine, arginine, can be included as 0-0.05% polysorbate-80 solution (optimally 0.005-0.01%). Additional surfactants include but are not limited to polysorbate-20 and BRIJ surfactants.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies and antibody portions of the present invention can be administered by a variety of methods known in the art, although for many therapeutic applications, the preferred route/mode of administration is subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Robinson, ed., Sustained and Controlled Release Drug Delivery Systems, Marcel Dekker, Inc., New York, 1978.

In certain embodiments, an antibody or antibody portion of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain embodiments, an antibody or antibody portion of the invention is coformulated with and/or coadministered with one or more additional therapeutic agents that are useful for treating diabetes mellitus and obesity. Furthermore, one or more antibodies of the invention may be used in combination with two or more of the commercially available therapeutic agents including, but not limited to sulfonylureas, metformin, thiazolidinediones (TZD's), acarbose and fenofibrate. Such combination therapies may advantageously utilize lower dosages of the administered therapeutic agents, thus avoiding possible toxicities or complications associated with the various monotherapies.

The antigens utilized to prepare the agonist antibodies (or allosteric modulator antibodies) of the present invention may be made via several possible methods. These methods are presented in detail, as follows:

Possible Antigens Utilized to Create The Agonistic Antibodies:

A. Cyclic Peptides Mimicking a Structural Loop

In one embodiment of the present invention, the agonistic antigen comprises a cyclic molecule, preferably a cyclic peptide, which structurally mimics a key loop of a common fold of the receptor against which antibodies are to be raised. To prepare this type of antigen, the structures of the extracellular loops of the GLP-1 receptor are simulated by synthetic peptides. Standard molecular modeling and crystallographic analysis can be used to aid in the identification of such looped extracellular domains. A linear molecule, e.g., a linear peptide, is designed based on these extracellular regions, and this linear molecule can then be cyclized, by known chemical means, to create an antigen that mimics the key loop. For example, a proline and a glycine can be added to a linear peptide to allow for cyclization of the peptide. A prominent loop on the HIV coat protein GP120 called V3 displays this beta-turn inducing feature. These beta turns exploit the natural geometry of a glycine-proline dyad to form a 180 degree turn that loops back onto itself. (See Vranken et al. (1996), European Journal of Biochemistry 236:100-108.) Such loops have been identified as being immunologically dominant sites for antibody CDR binding and experimentally serve as immunologically preferred “targets”. (See Moore et al. (1995) Journal of Virology 69:122-130.)

B. Hybrid Molecules

In another embodiment of the present invention, an agonistic antigen comprises a hybrid molecule, preferably a hybrid peptide that includes regions of the GLP-1 receptor and another protein that can present the loops and extracellular domain in an antigenic manner. (By “antigenic manner”, it is implied that a previously non-immunogenic region of the GLP-1 receptor is made immunogenic by its display within a foreign protein.) This may be useful in a situation where the human receptor is not immunogenic to a mouse that contains the humanized immune repertoire. To prepare this type of antigen, the structures of the two molecules are compared, and homologous regions are aligned. For example, an alignment of TSH receptor and GLP-1 receptor can be established in a way where extracellular loops 1,2,3 from GLP-1 receptor are then used to replace the extracellular loops 1,2,3 from TSH receptor. Since TSH receptor has an established proclivity to elicit agonist antibodies in patients with Graves' disease, this hybrid molecule receptor may facilitate the production of agonist antibodies to the domains of the GLP-1 receptor, which might otherwise not be immunogenic. This example defines the functional use of a hybrid molecule (e.g., a hybrid peptide, in which the two related molecules are proteins) that preferably comprises alternating regions (e.g., amino acid sequences) from each of the two molecules, interspersed with sections from the other molecule. An example of this would be a peptide where the extracellular loops are derived from a partial sequence of the GLP-1 receptor, and the transmembrane domains and intracellular loops are derived from another GPCR like glucagon or TSH receptor.

Another type of hybrid molecule is one in which a peptide has been introduced into a full-length protein (referred to as a “target protein”). Specific extracellular domains of one receptor are introduced into the full-length protein of the other related protein or, alternatively, an unrelated protein. For example, a peptide loop from human GLP-1 receptor corresponding to extracellular loop 1 is introduced into the full-length TSH receptor generating a foreign hybrid GLP-1 receptor/TSH receptor molecule. This introduction of the functional peptide into the related full-length protein constrains the functional peptide at both ends and maintains the fold structure of the functional peptide.

In the case of GLP-1 extracellular domains, a peptide preferably is inserted (i.e., replaces the natural amino acids) in a target area representing the analogous extracellular loops of the TSH receptor or glucagon receptor. Such areas may be found over the entire length of the protein (e.g., in the amino-terminal region, in the middle of the protein, in the carboxy-terminal region). Furthermore, extracellular peptides representing the GLP-1 receptor can also be inserted into an irrelevant protein, such as KLH (keyhole limpet hemocyanin) or some other naturally occurring or synthetic protein. In this instance, the preferred insertion site for the peptide is a region that allows the peptide to maintain the desired fold structure. Therefore, the insertion sites can be either at the amino-terminus, the middle, or the carboxy-terminus of the protein. The positioning of the peptide in any naturally occurring target protein is selected to mimic the structural constraints placed upon it by the native protein from which it is derived. While the antigen peptide may simply be inserted into the target protein such that the amino acids of the functional peptide are added to the target protein, preferably the amino acids of the peptide replace a portion of the target protein into which it is inserted. This molecule can be prepared using standard molecular biology techniques (e.g., cloning, polymerase chain reaction) using the publicly available human GLP-1 receptor and human TSH receptor cDNA sequences and recombinant protein expression techniques. A hybrid cDNA can be prepared, introduced into an appropriate expression vector, and the polypeptide can be expressed by introducing the expression vector into an appropriate host cell (see Sambrook and Russell, Molecular Cloning; A Laboratory Manual, Third Edition, Cold Spring Harbor Laboratory, Cold Spring Harbor, N.Y., 2001; Ausubel et al. (eds.) Current Protocols in Molecular Biology, Greene Publishing Associates, 1989). An example of heterologous epitope display using a hybrid molecule is exemplified in studies designed to graft foreign viral loops on the surface of E. coli alkaline phosphatase to serve as a target for monoclonal antibodies. (See Brennan et al. (1995) Proceedings of the National Academy of Science USA 92:5783-5787.) Receptor domain swapping between very similar GPCRs is described in Runge et al. (2003) British Journal of Pharmacology 138: 787-794.)

C. Antigen-Transfected Cells

Additionally, cell lines may be generated that stably express the agonistic antigens, or a hybrid molecule thereof. For example, cell lines can be generated that stably or transiently express either an intact human GLP-1 receptor or a hybrid molecule. The molecules of interest can be either secreted from the cells with addition of secretion signal peptides (in the case of soluble proteins) or can be expressed on the cell surface (in the case of receptors or enzymes). Gene delivery into the host cells, to allow for expression of the antigens by the host cells, can be accomplished by a number of conventional means, including but not limited to transfection, electroporation, cell fusion, lipofection, particle bombardment, microinjection or viral infection. The cell lines expressing the antigens of interest can then be injected via one or more various routes (intraperitoneal, subcutaneous, intramuscular, and the like) into an animal of interest for antibody production. The cells then serve as a slow release source of the antigen of interest. Preferably, the cells express the full-length proteins. However, antigenic fragments can also be expressed. To generate an agonistic antibody to the extracellular domain of GPCRS, the receptors preferably are expressed on the cell surface and verified using fluorescence activated cell sorting. (See Costagliola et al. (2002) supra.)

D. Immunization with Genetic Immunization Vectors

In an additional embodiment of the present invention, the agonistic antigen is simply encoded in a DNA vector capable of expressing the encoded gene in a living animal. The DNA vector itself is used as the immunization agent, and then the resultant antibody repertoire is screened for antibodies that bind and, more preferably, activate the GLP-1 receptor. For example, one can immunize a XenoMouse® (Abgenix, Fremont, Calif.) with a vector containing the human GLP-1 receptor, then screen for antibodies that bind to CHO cells expressing the human GLP-1 receptor, but not CHO cells expressing an unrelated GPCR receptor, and more preferably, antibodies that induce activation of the receptor. (As used herein, the term “immunize” is intended to broadly encompass the exposure of an antibody repertoire to the in vivo expressed antigen like a full-length GLP-1 receptor or a hybrid GLP-1 receptor which has domains switched with other receptors.)

As noted above, once the antigen has been made, one must then raise antibodies to the antigen. This may be accomplished via several methods, as presented in detail below:

Methods of Making Agonist Antibodies

To prepare an agonist antibody of the present invention, an antibody repertoire (either in vivo or in vitro) is exposed to an agonistic antigen, prepared as described in the previous section, and an appropriate agonistic antibody is selected from the repertoire. The two elements of antibody recognition of an antigen are structural recognition and affinity maturation based on specific molecular interactions. During a natural immune response, low affinity antibodies that recognize structural motifs (for example, the recognition of an antigen by certain pattern recognition receptors) are developed readily, and early on in the natural immune response, this is followed by somatic mutations to increase the affinity of several clones. Various in vivo and in vitro processes have been developed to mimic this natural phenomenon. Low affinity agonist antibodies can be generated by any of the in vitro and in vivo methods described herein and higher affinity agonist monoclonal antibodies can be prepared by somatic mutagenesis methods described herein. Moreover, to optimize high affinity dual specificity monoclonal antibodies, co-crystal structures of the low affinity monoclonal antibodies with the desired antigens can be made. The structural information obtained can guide further affinity enhancements by altering (mutating) specific contact residues of the monoclonal antibodies to enhance specific molecular interactions, as described herein.

Methods for making agonist antibodies of the present invention, using in vivo approaches, in vitro approaches, or a combination of both, are described in further detail in the following subsections.

A. In vivo Approaches

A standard in vivo approach to preparing antibodies is by immunizing an appropriate animal subject with an antigen to thereby expose the in vivo antibody repertoire to the antigen, followed by recovery of an antibody or antibodies of interest from the animal. Such an approach can be adapted to the preparation of agonistic antibodies by use of an agonistic antigen and selection for antibodies that specifically activate the receptor of interest. Agonistic antibodies can be prepared by immunizing a suitable subject, (e.g., rabbit, goat, mouse or other mammal, including transgenic and knockout versions of such mammals) with an immunogenic preparation of agonistic antigen. An appropriate immunogenic preparation can contain, for example, a chemically synthesized or recombinantly expressed agonistic antigen. Other alternative agonistic antigens include previously discussed transfected mammalian cells and genetic immunization. The preparation can further include an adjuvant, such as Freund's complete or incomplete adjuvant, or similar immunostimulatory compound. Moreover, when used to raise antibodies, in particular by in vivo immunization, an agonistic antigen of the invention can be used alone, or more preferably, as a conjugate with a carrier protein. Such an approach for enhancing antibody responses is well known in the art. Examples of suitable carrier proteins to which an antigen can be conjugated include KLH and albumin.

Antibody-producing cells can be obtained from the subject and used to prepare monoclonal antibodies by standard techniques, such as the hybridoma technique originally described by Kohler and Milstein ((1975) Nature supra; see also, Brown et al. (1981) Journal of Immunology 127:539-546; Brown et al. (1980) Journal of Biological Chemistry 255:4980-4983; Yeh et al. (1976) Proceedings of the National Academy of Science USA 76:2927-2931; and Yeh et al. (1982) International Journal of Cancer 29:269-275). The technology for producing monoclonal antibody hybridomas is well known (see generally Kenneth, in Monoclonal Antibodies: A New Dimension In Biological Analyses, Plenum Publishing Corp., New York, N.Y., 1980; Lerner (1981) Yale Journal of Biology and Medicine 54:387-402; Gefter et al. (1977) Somatic Cell Genetics 3:231-236). Briefly, an immortal cell line (typically a myeloma) is fused to lymphocytes (typically splenocytes or lymph node cells or peripheral blood lymphocytes) from a mammal immunized with an antigen as described above, and the culture supernatants of the resulting hybridoma cells are screened to identify a hybridoma producing a monoclonal antibody with agonistic activity for the GLP-1 receptor. Any of the many well known protocols used for fusing lymphocytes and immortalized cell lines can be applied for the purpose of generating agonistic monoclonal antibodies. (See, e.g., Galfre et al. (1977) Nature 266:550-552; Gefter et al. (1977) supra; Lerner (1981) supra; Kenneth, 1980, supra). Moreover, the ordinary skilled artisan will appreciate that there are many variations of such methods, which also may be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). A myeloma cell line (e.g., the P3-NS1/1-Ag4-1, P3-x63-Ag8.653 or Sp2/0-Ag14 myeloma lines) may be employed as a fusion partner according to standard hybridoma techniques. Aforementioned myeloma lines are available from the American Type Culture Collection (ATCC) in Manassas, Va. Typically, HAT-sensitive mouse myeloma cells are fused to mouse splenocytes using polyethylene glycol (“PEG”). Hybridoma cells resulting from the fusion are then selected using HAT medium, which selectively kills unfused and incompatibly fused myeloma cells (unfused splenocytes are lost after several days since they are not immortalized). Hybridoma cells producing monoclonal antibodies that specifically recognize the GLP-1 receptor or hybrid molecular variants thereof are identified by screening the hybridoma culture supernatants for such antibodies, e.g., using a standard ELISA assay, to select those antibodies that specifically can bind the target molecules.

Depending on the type of antibody desired, various animal hosts may be used for in vivo immunization. A host that itself expresses an endogenous version of the antigen(s) of interest can be used or, alternatively, a host can be used that has been rendered deficient in an endogenous version of the antigen(s) of interest. For example, it has been shown that mice rendered deficient for a particular endogenous protein via homologous recombination at the corresponding endogenous gene (i.e., “knockout” mice) elicit a humoral response to the protein when immunized with it and thus can be used for the production of high affinity monoclonal antibodies to the protein. (See, e.g., Roes et al. (1995) Journal of Immunological Methods 183:231-237; Lunn et al. (2000) Journal of Neurochemistry 75:404-412.)

For production of nonhuman antibodies (e.g., against a human agonistic antigen), various nonhuman mammals are suitable as hosts for antibody production, including but not limited to mice, rats, rabbits and goats (and knockout versions thereof); although, mice are preferred for hybridoma production. Furthermore, for production of fully human antibodies against a human agonistic antigen, a host nonhuman animal can be used that expresses a human antibody repertoire. Such nonhuman animals include transgenic animals (e.g., mice) carrying human immunoglobulin transgenes, hu-PBMC-SCID chimeric mice, and human/mouse radiation chimeras, each of which is discussed further below.

In one method of the present invention, the animal that is immunized with an agonistic antigen is a nonhuman mammal, preferably a mouse, that is transgenic for human immunoglobulin genes such that the nonhuman mammal (e.g., mouse) makes human antibodies upon antigenic stimulation. In such animals, typically, human germline configuration heavy and light chain immunoglobulin transgenes are introduced into animals that have been engineered so that their endogenous heavy and light chain loci are inactive. Upon antigenic stimulation of such animals (e.g., with an agonistic antigen), antibodies derived from the human immunoglobulin sequences (i.e., human antibodies) are produced, and human monoclonal antibodies can be made from lymphocytes of such animals by standard hybridoma technology. For a further description of human immunoglobulin transgenic mice and their use in the production of human antibodies see, for example, U.S. Pat. No. 5,939,598, U.S. Pat. No. 6,150,584, International Application Publication No. WO 96/34096, International Application Publication No. WO 98/24893, International Application Publication No. WO 99/53049, U.S. Pat. No. 5,545,806, U.S. Pat. No. 5,569,825, U.S. Pat. No. 5,625,126, U.S. Pat. No. 5,633,425, U.S. Pat. No. 5,661,016, U.S. Pat. No. 5,770,429, U.S. Pat. No. 5,814,318, U.S. Pat. No. 5,877,397 and U.S. Pat. No. 6,255,458. See also MacQuitty and Kay (1992) Science 257:1188; Taylor et al. (1992) supra; Lonberg et al. (1994) Nature 368:856-859; Lonberg and Huszar (1995) International Reviews of Immunology 13:65-93; Harding and Lonberg (1995) Annals of the NY Academy of Science 764:536-546; Fishwild et al. (1996) Nature Biotechnology 14:845-851; Mendez et al. (1997) Nature Genetics 15:146-156; Green and Jakobovits (1998) Journal of Experimental Medicine 188:483-495; Green (1999) Journal of Immunological Methods 231:11-23; Yang et al. (1999) Journal of Leukocyte Biology 66:401-410; Gallo et al. (2000) European Journal of Immunology 30:534-540.

In an alternative method of the present invention, the animal that is immunized with an agonistic antigen is a mouse with severe combined immunodeficiency (SCID) that has been reconstituted with human peripheral blood mononuclear cells or lymphoid cells or precursors thereof. Such mice, referred to as hu-PBMC-SCID chimeric mice, have been demonstrated to produce human immunoglobulin responses upon antigenic stimulation. For further description of these mice and their use in antibody generation, see for example Leader et al. (1992) Immunology 76:229-234; Bombil et al. (1996) Immunobiology 195:360-375; Murphy et al. (1996) Seminars in Immunology 8:233-241; Herz et al. (1997) International Archives of Allergy and Immunology 113:150-152; Albert et al. (1997) Journal of Immunology 159:1393-1403; Nguyen et al. (1997) Microbiology and Immunology 41:901-907; Arai et al. (1998) Journal of Immunological Methods 217:79-85; Yoshinari and Arai (1998) Hybridoma 17:41-45; Hutchins et al. (1999) Hybridoma 18:121-129; Murphy et al. (1999) Clinical Immunology 90:22-27; Smithson et al. (1999) Molecular Immunology 36:113-124; Chamat et al. (1999) Journal of Infectious Diseases 180:268-277; and Heard et al. (1999) Molecular Medicine 5:35-45.

In a further method, the animal that is immunized with an agonistic antigen is a mouse that has been treated with lethal total body irradiation, followed by radioprotection with bone marrow cells of a severe combined immunodeficiency mouse, followed by engraftment with functional human lymphocytes. This type of chimera, referred to as the Trimera system (Reisner and Dagan (1998) Trends in Biotechnology 16:242-246) has been used to produce human monoclonal antibodies by immunization of the mice with an antigen of interest followed by preparation of monoclonal antibodies using standard hybridoma technology. For further description of these mice and their use in antibody generation, see, for example, Eren et al. (1998) Immunology 93:154-161; Ilan et al. (1999) Hepatology 29:553-562; and Bocher et al. (1999) Immunology 96:634-641.

B. In vitro Approaches

As an alternative to preparing agonistic antibodies by in vivo immunization and selection, an agonistic antibody of the invention can be identified and isolated by screening a recombinant combinatorial immunoglobulin library (e.g., an antibody phage display library) with an agonistic antigen, to thereby isolate immunoglobulin library members that bind specifically to the GLP-1 receptor. Kits for generating and screening phage display libraries are commercially available (e.g., the Amersham Biosciences-GE Healthcare Recombinant Phage Antibody System (RPAS), RPAS. Mouse ScFv Module Catalog No. 27-9400-01. In various embodiments, the phage display library is a scFv library or a Fab library. The phage display technique for screening recombinant antibody libraries has been described extensively in the art. Examples of methods and compounds particularly amenable for use in generating and screening antibody display library can be found in, for example, McCafferty et al. International Publication No. WO 92/01047, U.S. Pat. No. 5,969,108 and EP 589,877 (describing, in particular, display of scFv), Ladner et al., U.S. Pat. No. 5,223,409, U.S. Pat. No. 5,403,484, U.S. Pat. No. 5,571,698, U.S. Pat. No. 5,837,500 and EP 436,597 (describing, for example, pIII fusion); Dower et al., International Publication No. WO 91/17271, U.S. Pat. No. 5,427,908, U.S. Pat. No. 5,580,717 and EP 527,839 (describing, in particular, display of Fab); Winter et al., International Publication WO 92/20791 and EP 368,684 (describing, in particular, cloning of immunoglobulin variable domain sequences); Griffiths et al., U.S. Pat. No. 5,885,793 and EP 589,877 (describing, in particular, isolation of human antibodies to human antigens using recombinant libraries); Garrard et al., International Publication No. WO 92/09690 (describing, in particular, phage expression techniques); Knappik et al. International Publication No. WO 97/08320 (describing the human recombinant antibody library HuCal); Salfeld et al., International Publication No. WO 97/29131, describing the preparation of a recombinant human antibody to a human antigen (human tumor necrosis factor alpha), as well as in vitro affinity maturation of the recombinant antibody) and Salfeld et al., U.S. Provisional Patent Application No. 60/126,603, also describing the preparation of a recombinant human antibody to a human antigen (human interleukin-12), as well as in vitro affinity maturation of the recombinant antibody).

Other descriptions of recombinant antibody library screenings can be found in scientific publications such as Fuchs et al. (1991) Bio/Technology 9:1370-1372; Hay et al. (1992) Human Antibody Hybridomas 3:81-85; Huse et al. (1989) Science 246:1275-1281; Griffiths et al. (1993) EMBO Journal 12:725-734; Hawkins et al. (1992) Journal of Molecular Biology 226:889-896; Clarkson et al. (1991) Nature 352:624-628; Gram et al. (1992) Proceedings of the National Academy of Science USA 89:3576-3580; Garrad et al. (1991) Bio/Technology 9:1373-1377; Hoogenboom et al. (1991) Nucleic Acids Research 19:4133-4137; Barbas et al. (1991) Proceedings of the National Academy of Science USA 88:7978-7982; McCafferty et al. (1990) Nature 348:552-554; and Knappik et al. (2000) Journal of Molecular Biology 296:57-86.

Alternative to the use of bacteriophage display systems, recombinant antibody libraries can be expressed on the surface of yeast cells or bacterial cells. Methods for preparing and screening libraries expressed on the surface of yeast cells are described further in International Application Publication No. WO 99/36569. Methods for preparing and screening libraries expressed on the surface of bacterial cells are described further in U.S. Pat. No. 6,699,658.

Once an antibody of interest has been identified from a combinatorial library, DNAs encoding the light and heavy chains of the antibody are isolated by standard molecular biology techniques, such as by polymerase chain reaction (PCR) amplification of DNA from the display package (e.g., phage) isolated during the library screening process. Nucleotide sequences of antibody light and heavy chain genes from which oligonucleotide primers can be prepared are known in the art. For example, many such sequences are disclosed in Kabat et al. (1991) supra and in the “Vbase” human germline sequence database, administered by the MRC Centre for Protein Engineering (Cambridge, UK) (website: http://www.mrc-cpe.cam.ac.uk/vbase-intro.php?menu=901).

An antibody (or portion thereof) of the invention may be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook and Russell (2001) supra; Ausubel (1989) supra; and in U.S. Pat. No. 4,816,397.

Once DNA fragments encoding the VH and VL segments of the antibody of interest are obtained, these DNA fragments can be further manipulated by standard recombinant DNA techniques, for example, to convert the variable region genes to full-length antibody chain genes, to Fab fragment genes or to a ScFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see, e.g., Kabat et al. (1991) supra) and DNA fragments encompassing these regions can be obtained by standard polymerase chain reaction amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat et al. (1991) supra) and DNA fragments encompassing these regions can be obtained by standard polymerase chain reaction amplification. The light chain constant region can be a kappa or lambda constant region, but most preferably is a kappa constant region.

To create a scFv gene, the VH- and VL-encoding DNA fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see e.g., Bird et al. (1988) supra; Huston et al. (1988) supra; McCafferty et al. (1990) supra).

To express the recombinant antibodies, or antibody portions of the invention, DNAs encoding partial or full-length light and heavy chains, obtained as described above, can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). Prior to insertion of the light or heavy chain sequences, the expression vector may already carry antibody constant region sequences. For example, one approach to converting the VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino-terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology: Methods in Enzymology, Vol. 185, pp. 3-7, Academic Press, San Diego, Calif., 1990). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062, U.S. Pat. No. 4,510,245 and U.S. Pat. No. 4,968,615.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in DHFR⁻ host cells with methotrexate selection/amplification) and the neomycin resistance gene (for G418 selection).

For expression of the light and heavy chains, the expression vector(s) encoding the heavy and light chains is/are transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, lipid-mediated DNA transfer and the like. Although it is theoretically possible to express the antibodies of the invention in either prokaryotic or eukaryotic host cells, expression of antibodies preferably in eukaryotic cells, and most preferably mammalian host cells, is the most preferred method because such eukaryotic cells and, in particular, mammalian cells, are more likely than prokaryotic cells to assemble and secrete a properly folded and immunologically active antibody. Prokaryotic expression of immunoglobulin has been reported to be suboptimal for high yield production of active antibody (Boss and Wood (1985) Immunology Today 6:12-13; Fernandez (2004) Current Opinion in Biotechnology 15:364-373).

Preferred mammalian host cells for expressing the recombinant antibodies of the invention include Chinese Hamster Ovary (CHO) cells (including DHFR⁻ CHO cells, described in Urlaub and Chasin (1980) Proceedings of the National Academy of Science USA 77:4216-4220, used with a DHFR selectable marker, e.g., as described in Kaufman and Sharp (1982) Journal of Molecular Biology 159:601-621), NS0 myeloma cells, COS cells and SP2/0-Ag14 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. It will be understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to the antigens of interest. The molecules expressed from such truncated DNA molecules are also encompassed by the antibodies of the invention. In addition, agonist antibodies may be produced in which one heavy and one light chain are an antibody of the invention and the other heavy and light chain are specific for one epitope on the same receptor of interest by cross-linking an antibody of the invention to a second antibody by standard chemical cross-linking methods. Such cross-linked antibody dyads could confer special agonist properties to the conjugation product that is unattainable with the individual antibodies.

In a preferred system for recombinant expression of an antibody, or antigen-binding portion thereof, of the invention, a recombinant expression vector encoding both the antibody heavy chain and the antibody light chain is introduced into DHFR⁻ CHO cells by calcium phosphate-mediated transfection or by other commercially available transfection reagents such as FuGENE6 (Roche, Indianapolis, Ind.) or Lipofectamine™ (Invitrogen Corporation, Carlsbad, Calif.). Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection and amplification. The selected transfected host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. Still further the invention provides a method of synthesizing a recombinant antibody of the invention by culturing a host cell of the invention in a suitable culture medium until a recombinant antibody of the invention is synthesized. The method can further comprise isolating the recombinant antibody from the culture medium.

Alternative to screening of recombinant antibody libraries by phage display, other methodologies known in the art for screening large combinatorial libraries can be applied to the identification of agonist antibodies of the invention. One type of alternative expression system is one in which the recombinant antibody library is expressed as RNA-protein fusions, as described in International Application Publication No. WO 98/31700 and Austrian Patent No. 738328 by Szostak and Roberts, and in Roberts and Szostak (1997) Proceedings of the National Academy of Science USA 94:12297-12302. In this system, a covalent fusion is created between a mRNA and the peptide or protein that it encodes by in vitro translation of synthetic mRNAs that carry puromycin, a peptidyl acceptor antibiotic, at their 3′-end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the agonistic antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above.

C. Combination Approaches

Agonistic antibodies of the invention also can be prepared using a combination of in vivo and in vitro approaches, such as methods in which the agonistic antigen is originally exposed to an antibody repertoire in vivo in a host animal to stimulate production of antibodies that bind the receptor but wherein further antibody selection and/or maturation (i.e., improvement) is accomplished using one or more in vitro techniques. Such a combination method involves first immunizing a nonhuman animal (e.g., a mouse, rat, rabbit, goat, or transgenic version thereof, or a chimeric mouse) with the agonist inducing antigen to stimulate an antibody response against the receptor, followed by preparation and screening of a phage display antibody library using immunoglobulin sequences from lymphocytes stimulated in vivo by exposure to the antigen. The first step of this combination procedure can be conducted as described in subsection IIA above (in vivo methods), while the second step of this procedure can be conducted as described in subsection IIB above (in vitro methods). Preferred methodologies for hyperimmunization of nonhuman animals followed by in vitro screening of phage display libraries prepared from the stimulated lymphocytes include those described by BioSite Inc., see e.g., U.S. Pat. No. 6,420,113, U.S. Pat. No. 5,427,908, U.S. Pat. No. 5,427,908 and U.S. Pat. No. 5,580,717.

Further, an additional combination method of the present invention involves first immunizing a nonhuman animal (e.g., a mouse, rat, rabbit, goat, or knockout and/or transgenic version thereof, or a chimeric mouse) with the agonistic antigen (or allosteric modulator antibody of the present invention) to stimulate an antibody response against the antigen and selection of lymphocytes that are producing antibodies having the desired activity (e.g., by screening hybridomas prepared from the immunized animals). The rearranged antibody genes from the selected clones are then isolated (by standard cloning methods, such as reverse transcriptase-polymerase chain reaction) and subjected to in vitro affinity maturation, to thereby enhance the binding properties of the selected antibody or antibodies. The first step of this procedure can be conducted as described in subsection IIA above, while the second step of this procedure can be conducted as described in subsection IIB above, in particular using in vitro affinity maturation methods such as those described in U.S. Pat. No. 6,258,562 and International Application Publication No. WO 00/56772.

In yet another combination method, recombinant antibodies are generated from single, isolated lymphocytes using a procedure referred to in the art as the selected lymphocyte antibody method (SLAM), as described in U.S. Pat. No. 5,627,052, International Application Publication No. WO 92/02551 and Babcock et al. (1996) Proceedings of the National Academy of Science USA 93:7843-7848. In this method, as applied to the agonist antibodies or allosteric modulator antibodies of the invention, a nonhuman animal (e.g., a mouse, rat, rabbit, goat, or transgenic version thereof, or a chimeric mouse) first is immunized in vivo with the agonist-inducing antigen or allosteric modulator-inducing antigen to stimulate an antibody response against the antigen, and then single cells secreting antibodies of interest, e.g., specific for the agonistic antigen, are selected using an antigen-specific hemolytic plaque assay (e.g., the agonistic or allosteric modulator-inducing antigen itself), is coupled to sheep red blood cells using a linker, such as biotin, thereby allowing for identification of single cells that secrete antibodies with the appropriate specificity using the hemolytic plaque assay). Following identification of antibody-secreting cells of interest, heavy- and light-chain variable region cDNAs are rescued from the cells by reverse transcriptase-polymerase chain reaction and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example, by panning the transfected cells to isolate cells expressing antibodies having the desired agonistic specificity. The amplified immunoglobulin sequences further can be manipulated in vitro, such as by in vitro affinity maturation, as described above.

Methods for Making Allosteric Modulatory Antibodies

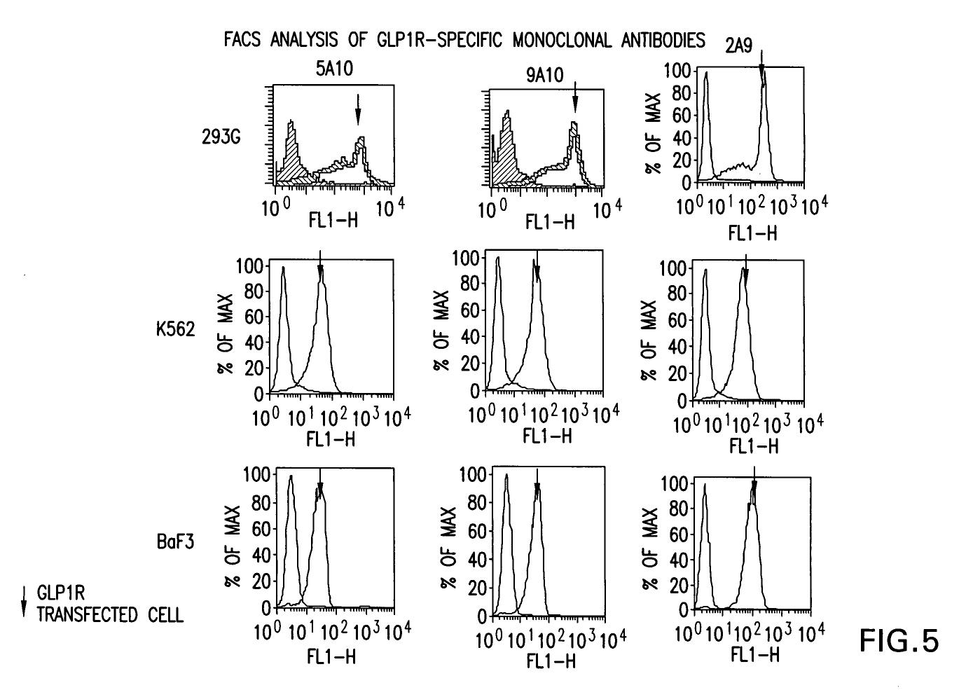

Methods described in detail above are applicable towards the generation of allosteric modulator antibodies of the invention. Further, methods and protocols described to develop and utilize variant immunogens capable of eliciting the formation of agonist antibodies are likewise capable of developing allosteric antibodies against the GLP-1 receptor. Also, methods employing multiple immunizations of wild type or transgenically humanized mice with fully intact or truncated receptor domains of the human GLP-1 receptor have successfully induced positive seroconversion of animal sera extracted from these animals in ELISA assays against GLP1R. Harvested spleens from seropositive animals were then utilized to develop hybridomas employing techniques as described above in the section “in vivo approaches” of making antibodies. Derivative supernatants from stable hybridomas were tested in ELISA assays to verify ability of encoded antibodies to bind GLP1R. Corroborative florescence activated cell sorter (FACS) binding studies conducted on live cells expressing high copy numbers of receptors in a native configuration were performed in parallel for some candidate hybridomas as described in FIG. 5 and described under Example 4. FACS analysis of candidate antibodies were performed against three different cell types either expressing or not expressing GLP1R to verify selectivity of antibody paratopes for the target receptor. Details of protocols for this analysis are included in Example 4.

Agonist-Specific vs. Allosteric Antibody Screening

Functional assays designed to identify antibodies with pure agonist activity are performed in the complete absence of any peptide agonist. Candidate antibodies or derivative hybridomas are assayed with reporter cells incubated as described. Assays designed to detect allosteric antibodies are performed in the presence of submaximal concentrations of GLP-1 ranging from 10-30 picomolar to simulate physiological levels of incretin.

Uses of Agonist and Allosteric Antibodies

Given their ability to bind and activate or modulate the GLP-1 receptor, the agonist and allosteric antibodies of the present invention, respectively, or portions thereof, may be used to detect the presence of the receptor (e.g., in a biological sample, such as prepared tissue or biopsy), using a conventional immunoassay, such as an enzyme linked immunosorbent assay (ELISA), a radioimmunoassay (RIA), or tissue immunohistochemistry. In particular, the invention provides a method for detecting an antigen in a biological sample comprising contacting a biological sample with an agonistic or allosteric antibody or portion thereof of the invention that specifically recognizes the antigen and detecting either the antibody (or antibody portion) bound to antigen or unbound antibody (or antibody portion), to thereby detect the antigen in the biological sample. The antibody is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride and phycoerythrin. An example of a luminescent material includes luminol, and examples of suitable radioactive material include ¹²⁵, ¹³¹I, ³⁵S or ³H.

Alternative to labeling the antibody, the antigen(s) can be assayed in biological fluids by a competition radioimmunoassay utilizing antigen standards labeled with a detectable substance and an unlabeled antibody specific for the antigen(s). In this assay, the biological sample, the labeled antigen standards and the agonistic or allosteric modulator antibody are combined, and the amount of labeled antigen standard bound to the unlabeled antibody is determined. The amount of antigen in the biological sample is inversely proportional to the amount of labeled antigen standard bound to the unlabeled antibody.

In a preferred embodiment, the agonistic or allosteric antibody specifically recognizes GLP-1 receptor and the foregoing detection methods are used to detect GLP-1 receptor. Accordingly, the invention further provides a method of detecting GLP-1 receptor in a biological sample or tissue comprising contacting the biological sample or tissue suspected of containing GLP-1 receptor with an agonistic or allosteric antibody of the invention, or antigen-binding portion thereof, and detecting GLP-1 receptor in the biological sample or tissue. The biological sample can be, for example, an in vitro sample, such as a sample of cells, tissue or bodily fluid (e.g., blood, pancreatic extract, urine, saliva, biopsy, etc.). Moreover, the tissue detected can be tissue located in vivo in a subject, e.g., tissue visualized by in vivo imaging of the tissue (e.g., using a labeled antibody). Such an application can be employed to estimate the size and density of islet tissue in a living human subject, thereby, estimating the efficacy of treatment for diabetic-related apoptosis of islets of Langerhans.

The GLP-1 receptor agonistic or allosteric antibodies of the invention also can be used for diagnostic purposes. In one example a certain unspecified tumor may present high copies of the GLP-1 receptor (e.g., in neuroendocrine tumors as described by Reubi and Waser in European Journal of Nuclear Medicine and Molecular Imaging (2003) 30:781-793 or hepatoma or pancreatic neoplastic cells). In this embodiment, an antibody of the invention is used in a diagnostic assay in vitro, such as in a laboratory test to detect the antigen(s) of interest or in a point of care test to detect the antigen(s) of interest from a hepatic or pancreatic biopsy. Examples of well-established in vitro assays utilizing antibodies include ELISAs, RIAs, Western blots and the like. In another embodiment, an antibody of the invention is used in a diagnostic assay in vivo, such as an in vivo imaging test. For example, the antibody can be labeled with a detectable substance capable of being detected in vivo, the labeled antibody can then be administered to a subject, and the labeled antibody can be detected in vivo, thereby allowing for in vivo imaging.

In another embodiment, the invention provides a method for activating receptor activity in a subject suffering from a disorder in which that activity is beneficial. In particular, the invention provides methods for activating or modulating the GLP-1 receptor in a subject suffering from diabetes or obesity which method comprises administering to the subject the agonist or allosteric antibody, or portion of the antibody, invention such that receptor activity in the subject is enhanced or modulated, as appropriate. Preferably, the receptor is a human GLP-1 receptor and the subject is a mammal and, in particular, a human subject. An antibody of the invention can be administered to a human subject for therapeutic purposes. Moreover, an antibody of the invention can be administered to a nonhuman mammal expressing the GLP-1 receptor with which the antibody binds for veterinary purposes or as an animal model of human disease. Regarding the latter, such animal models may be useful for evaluating the therapeutic efficacy of antibodies of the invention (e.g., testing of dosages and time courses of administration).

The agonistic or allosteric antibody can be employed to attenuate the effects of apoptosis-mediated degenerative diseases of the central nervous system like Alzheimer's Disease, Creutzfeld-Jakob Disease and bovine spongiform encephalopathy, chronic wasting syndrome and other prion mediated apoptotic neural diseases (e.g., as reviewed by Perry and Grieg (2004) Current Drug Targets 6:565-571). Administration of the agonistic or allosteric modulator antibody may also lead to down-modulation of βAPP as demonstrated by Perry et al. (2003) Journal of Neuroscience Research 72:603-612 and thereby ameliorate Aβ mono- or oligomer-mediated pathologies associated with Alzheimer's Disease. The agonistic or allosteric modulator antibody may also be administered to improve learning and memory by enhancing neuronal plasticity and facilitation of cellular differentiation as observed by During et al. (2003) Nature Medicine 9:1173-1179. Further, the agonistic or allosteric modulator antibody may also be used to preserve dopamine neurons and motor function in Morbus Parkinson as demonstrated by Greig et al. (2005) Abstract 897.6, Society for Neuroscience, Washington, D.C.

The human or humanized antibodies of the invention, and portions of these antibodies, can be used to treat humans suffering from autoimmune diseases, in particular, those associated with inflammation, including, autoimmune diabetes, adult onset diabetes, morbid obesity, Metabolic Syndrome X and dyslipidemia. The GLP-1 receptor antibody can also be employed as a growth factor for the promotion of islet growth in persons with autoimmune diabetes. The agonistic antibody is also useful in the treatment of congestive heart failure.

A GLP-1 receptor agonistic or allosteric modulator antibody of the invention, or antibody portion, can be administered with one or more additional therapeutic agents useful in the treatment of diabetes, dyslipidemia, and other metabolic diseases.

Antibodies of the invention, or antigen binding portions thereof, can be used alone or in combination to treat such diseases. It should be understood that the antibodies of the invention, or antigen binding portion or portions thereof, can be used alone or in combination with an additional agent, e.g., a therapeutic agent, said additional agent being selected by the skilled artisan for its intended purpose. For example, the additional agent can be a therapeutic agent, art-recognized as being useful to treat the disease or condition being treated by the antibody of the present invention. The additional agent also can be an agent that imparts a beneficial attribute to the therapeutic composition e.g., an agent that affects the viscosity of the composition.

It should further be understood that the combinations which are to be included within this invention are those combinations useful for their intended purpose. The agents set forth below are for illustrative purposes and not intended to be limiting. For example, the combinations which are part of this invention can be the antibodies of the present invention and at least one additional agent selected from the lists below. The combination may also include more than one additional agent, e.g., two or three additional agents if the combination is such that the formed composition can perform its intended function.

Preferred combinations are oral antidiabetic agent (OAA) drug(s) also referred to as OAA which include drugs like metformin, acarbose, TZD's). Inhalable insulin represents a new mode and potentially widespread means of administering this drug. Well known side effects of insulin which include hypoglycemia, and its concomitant comatose endpoint, may be drastically reduced when supplanted with the agonist antibody of the invention. Non-limiting examples of therapeutic agents for diabetes with which an antibody, or antibody portion, of the invention can be combined include the following: acarbose, metformin, sufonylureas, TZD's, squalene synthase inhibitors, other lipid lowering agents and aspirin.

Metabolic Syndrome X is a combination of disorders that increases the probability of developing diabetes, cardiac disease, or vascular dysfunction. It is generally accompanied by a sequelae of symptoms including: central obesity (characterized by abdominal adiposity), atherogenic dyslipidemia (characterized by high triglycerides and low HDL cholesterol), elevated blood pressure (130/85 mm Hg or higher), insulin resistance or glucose intolerance, a prothrombotic state (exemplified by high fibrinogen or plasminogen activator inhibitor in the blood) and a proinflammatory state (e.g., elevated levels of proinflammatory cytokines or markers of inflammation-like C-reactive protein in the blood).

Metabolic Syndrome X may be characterized as an inflammatory disease so the agonistic antibody may be coupled to form a bifunctional antibody complex against adipokine related cytokines including but not limited to PAI-1, TNFα, IL-6, MIF and IL-1. This can be accomplished by chemically complexing one molecule of the GLP-1 receptor agonist or allosteric modulating antibody to a cytokine-neutralizing monoclonal antibody. The coupling can also be achieved by genetic engineering methodology.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody or antibody portion may vary according to factors such as the disease state, age, sex, and weight of the individual, and the ability of the antibody or antibody portion to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimal desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit forms as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of an antibody or antibody portion of the invention is 0.1-20 mg/kg, more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Another embodiment of the invention provides a crystallized binding protein. Preferably the invention relates to crystals of whole agonistic antibodies and fragments thereof, as disclosed herein, and formulations and compositions comprising such crystals. In one embodiment, the crystallized binding protein has a greater half-life in vivo than the soluble counterpart of the binding protein. In another embodiment, the binding protein retains biological activity after crystallization.

Crystallized binding protein of the invention may be produced according methods known in the art and as disclosed in WO 02072636, incorporated herein by reference.

Another embodiment of the invention provides a glycosylated binding protein wherein the antibody or antigen-binding portion thereof comprises one or more carbohydrate residues. Nascent in vivo protein production may undergo further processing, known as post—translational modification. In particular, sugar (glycosyl) residues may be added enzymatically, a process known as glycosylation. The resulting proteins bearing covalently linked oligosaccharide side chains are known as glycosylated proteins or glycoproteins. Protein glycosylation depends on the amino acid sequence of the protein of interest, as well as the host cell in which the protein is expressed. Different organisms may produce different glycosylation enzymes (e.g., glycosyltransferases and glycosidases), and have different substrates (nucleotide sugars) available. Due to such factors, protein glycosylation pattern, and composition of glycosyl residues, may differ depending on the host system in which the particular protein is expressed. Glycosyl residues useful in the invention may include, but are not limited to, glucose, galactose, mannose, fucose, N-acetylglucosamine and sialic acid. Preferably the glycosylated binding protein comprises glycosyl residues such that the glycosylation pattern is human.

It is known to those skilled in the art that differing protein glycosylation may result in differing protein characteristics. For instance, the efficacy of a therapeutic protein produced in a microorganism host, such as yeast, and glycosylated utilizing the yeast endogenous pathway may be reduced compared to that of the same protein expressed in a mammalian cell, such as a CHO cell line. Such glycoproteins may also be immunogenic in humans and show reduced half-life in vivo after administration. Specific receptors in humans and other animals may recognize specific glycosyl residues and promote the rapid clearance of the protein from the bloodstream. Other adverse effects may include changes in protein folding, solubility, susceptibility to proteases, trafficking, transport, compartmentalization, secretion, recognition by other proteins or factors, antigenicity, or allergenicity. Accordingly, a practitioner may prefer a therapeutic protein with a specific composition and pattern of glycosylation, for example glycosylation composition and pattern identical, or at least similar, to that produced in human cells or in the species-specific cells of the intended subject animal.

Expressing glycosylated proteins different from that of a host cell may be achieved by genetically modifying the host cell to express heterologous glycosylation enzymes. Using techniques known in the art a practitioner may generate antibodies or antigen-binding portions thereof exhibiting human protein glycosylation. For example, yeast strains have been genetically modified to express non-naturally occurring glycosylation enzymes such that glycosylated proteins (glycoproteins) produced in these yeast strains exhibit protein glycosylation identical to that of animal cells, especially human cells (U.S. Patent Application Publication Nos. 20040018590 and 20020137134).

Further, it will be appreciated by one skilled in the art that a protein of interest may be expressed using a library of host cells genetically engineered to express various glycosylation enzymes, such that member host cells of the library produce the protein of interest with variant glycosylation patterns. A practitioner may then select and isolate the protein of interest with particular novel glycosylation patterns. Preferably, the protein having a particularly selected novel glycosylation pattern exhibits improved or altered biological properties.

Additional Uses of Allosteric Modulator Antibodies

The allosteric modulator antibody of the present invention, or portion thereof, may improve the sensitivity of the GLP-1 receptor such that full physiological activation is achieved in the presence of an endogenous or exogenous agonist concentration of 1000 picomolar or less, preferably with an agonist concentration of 10 picomolar or less, more preferably with an agonist concentration of 5 picomolar or less, even more preferably with an agonist concentration of 1 picomolar or less, even more preferably with an agonist concentration of 0.1 picomolar or less, and most preferably with an agonist concentration of 0.05 picomolar or less. (All integer concentrations between 1000 picomolar and 0.01 micromolar, as well as percentages thereof, are considered to fall within the scope of the invention.)

Further, the allosteric modulator antibodies of the present invention, or portions thereof, may be utilized in connection with cyclic AMP assays which are designed to quantitate Gs coupled receptor function by directly measuring amount of cAMP produced in proportion to level of receptor activity. Thus, an allosteric modulator antibody of the present invention, or portion thereof, may magnify the response of the GLP-1 receptor such that the resultant in vitro intracellular cyclic AMP response to agonist is increased by 5% or more for a given concentration of agonist, preferably such that the resultant in vitro intracellular cyclic AMP response to agonist is increased by 25% or more, more preferably such that the resultant in vitro intracellular cyclic AMP response to agonist is increased by 75% or more, even more preferably such that the resultant in vitro intracellular cyclic AMP response to agonist is increased by 100% or greater, even more preferably such that the resultant in vitro intracellular cyclic AMP response to agonist is increased by 200% or greater, and most preferably such that the resultant in vitro intracellular cyclic AMP response to agonist is increased by 400% or greater for every given concentration of agonist. (All integer percentages between 5% and 400%, as well as percentages between full integers, are considered to fall within the scope of the invention.)

Additionally, the allosteric modulator antibodies of the present invention, or portions thereof, may be used in connection with intracellular calcium release analysis. Intracellular calcium release is one mechanism by which receptors can transduce a signaling event into cellular function. It is currently employed as a rapid method to measure ligand-induced effects on GPCRs by combining a cell-permeable fluorophore that becomes fluorescent only upon calcium binding and an instrument configured to measure the fluorescent signal in real time (e.g. a Fluorometric Imaging Plate Reader—FLIPR). These instruments can evaluate intracellular calcium release commensurate with receptor activity and can capture quantitatively an increased magnitude in receptor activation. Cell lines that have been transfected with GLP-1 receptor and a promiscuous G protein (Gα-16) display coupled calcium release with receptor activation. Thus, an allosteric modulator antibody of the present invention, or portion thereof, may magnify the response of the GLP-1 receptor such that the resultant calcium release is 5% greater than the unmodulated receptor, preferably such that the resultant calcium release is 25% greater than the unmodulated receptor, more preferably such that the resultant calcium release is 75% greater than the unmodulated receptor, even more preferably such that the resultant calcium release is 200% greater than the unmodulated receptor, and most preferably such that the resultant calcium release is 400% greater than the unmodulated receptor. (All integer percentages between 5% and 400% are considered to fall within the scope of the invention as well as percentages thereof.)

It should also be noted that certain mammalian insulinoma cell lines (e.g. RIN-m5F, NIT-1, MIN-6, etc.), expressing GLP1R are responsive to GLP-1 peptide under cell culture conditions where glucose concentration is above 10 mM. GLP-1 induces accumulation of intracellular cAMP described earlier which mediates insulin secretion from these cells. Insulin secretion can be quantitated by ELISA assays (Linco Research Inc., St. Charles, Mo.) and thus be employed as a metric of GLP-1 receptor activation. An allosteric modulator antibody of the present invention, or portion thereof, may magnify the response of the GLP-1 receptor such that the resultant insulin release from insulinomas or harvested mammalian islets is 5% greater than the unmodulated receptor, preferably such that the resultant insulin secretion is 25% greater than the unmodulated receptor, more preferably such that the resultant calcium release is 100% greater than the unmodulated receptor, even more preferably such that the resultant calcium release is 200% greater than the unmodulated receptor, even more preferably such that the resultant calcium release is 400% greater than the unmodulated receptor. (All integer percentages between 5% and 400% are considered to fall within the scope of the present invention as well as percentages thereof.)

Further, it should be noted that increased GLP1R receptor function is postulated to result in improvements in symptoms associated with type 2 diabetes mellitus. Patients receiving DPP IV inhibitors experience a two-fold (100%) increase in circulating active GLP-1 resulting in clinically significant improvements in glucose control, HbA1_{c} and glucagon suppression (Ahren et al. (2004) Journal of Clinical Endocrinology and Metabolism 89: 2078-2084). An allosteric modulator antibody of the present invention, or portion thereof, may magnify the response of the GLP-1 receptor such that the resultant diabetic symptom is affected as follows: 1) plasma glucose level is lowered in vivo by 1 mM or greater, preferably such that the resultant treatment plasma glucose level is lowered in vivo by 2 mM or greater, more preferably such that the resultant treatment plasma glucose level is lowered in vivo by 3 mM or greater and most preferably such that the resultant treatment plasma glucose level is lowered in vivo by 5 mM or greater;

2) glycated hemoglobin designated as HbA1_{c} level is lowered in vivo to 7% or less in patients receiving antibody treatment, preferably such that the resultant HbA1_{c} level is lowered in vivo to 6% or less and more preferably such that the resultant HbA1_{c} level is lowered in vivo to 5% or less; and 3) glucagon secretion is suppressed by 10% as a function of increased GLP1R modulation in patients receiving antibody treatment, preferably such that the resultant in vivo glucagon secretion is suppressed by 50% or greater and more preferably such that the resultant in vivo glucagon secretion is suppressed by 80% or greater. Further, islet function is evaluated in in vivo animal models by microscopic staining of pancreatic sections for insulin secretory beta-cell and is predictive of improved islet function in patients receiving GLP1R antibody treatment. Stimulation of GLP1R in patients previously diagnosed with impaired islet function is predicted to improve islet repopulation and secretory function. Further, human plasma concentration of endogenous GLP-1 can range from 18-45 picomolar depending on the prandial state and meal size (Vilsbol et al. (2003) Journal of Clinical Endocrinology and Metabolism 88:2706-2713). Supra-physiological concentrations of agonist above this level have been exogenously introduced into subjects with marked improvement in therapeutic impact. Patient plasma glucose decreased by 5.5 mmol/L, hemoglobin A(1c)(HbA1_{c}) decreased by 1.3% and fructosamine levels were normalized. Fasting and 8 hr. mean concentrations of free fatty acids decreased by 30% and 23%. Gastric emptying was inhibited, resulting in average bodyweight reduction of 1.9 kg per subject, accompanied by a marked reduction in appetite. Both insulin sensitivity and beta-cell function improved. (Zander et al. (2002) Lancet 359:824-830.)

Analogous results are observed in patients receiving DPP IV inhibitors where a two-fold increase in circulating active GLP-1 results in dramatic improvements in glucose control, HbA1_{c} and glucagon suppression (Ahren et al. (2004) supra). It is postulated from these findings that minute increases in GLP-1 activation can translate into therapeutically significant disease-modifying improvements.

The present invention is further illustrated by the following examples which should not be construed as limiting in any way. Further, as noted previously, the contents of all cited references, including literature references, issued patents, and published patent applications, as cited throughout this application, are hereby expressly incorporated by reference. In particular, the present invention may be illustrated by the use of the following non-limiting examples:

### EXAMPLE 1

### Humanized Mouse/In Vitro Screening

Antigen

Abgenix (Fremont, Calif.) humanized mice are immunized against KLH-coupled peptides that mimic the three extracellular loops of the human GLP-1 receptor. A subset of these mice will be immunized against the peptides and the soluble extracellular amino-terminal domain of the GLP-1 receptor.

Peptides are synthesized on an ABI peptides synthesizer according to manufacturer's instructions.

| | Receptor | |
|---|---|---|
| | Extracel- | |
| Peptide | lular | |
| # | Domain | Peptide Sequence |
| 1 | EC1 | AALKWMYSTAAQQHQWDGLLSYQDSLS CRE |
| | | (SEQ ID NO: 7) |
| 2 | EC2 | CYEDEGCWTRNSNMNE |
| | | (SEQ ID NO: 8) |
| 3 | EC3 | CDEHARGTLRFIKLPTEE |
| | | (SEQ ID NO: 9) |

HPLC purified peptides are lyophilized and quantitated using both spectrophotometry and bicinchoninic acid (BCA) assay kits (Pierce Chemical Company, Rockford, Ill., Catalog No. 23227) after resuspension in phosphate buffered saline (Invitrogen Corporation, Carlsbad, Calif., Catalog No. 14040-117).
KLH-Conjugation of Peptides

Each peptide is conjugated to KLH using the Pierce Imject Maleimide Activated Immunogen Conjugation Kit (Pierce Chemical Company, Rockford, Ill., Catalog No. 77611) using the manufacturer's instructions. Briefly, each peptide is incubated with the activated KLH protein reagent and allowed to complete the reaction. Conjugated peptide is separated from free peptide using a desalting gel filtration column (Sephadex G50, Amersham Biosciences-GE Healthcare Piscataway, N.J.) then concentrated in a Centricon YM-30 protein concentrator (Millipore Corporation, Bedford, Mass., Catalog No. 4322).

Immunize Mice With KLH Conjugate

Each XenoMouse® (Abgenix, Fremont, Calif.) receives 20-50 micrograms of KLH-peptide conjugate suspended in 200 μl of Freund's complete adjuvant. Mice receive a booster innoculum three weeks after the initial immunization containing another 20-50 micrograms of conjugate in Freund's incomplete adjuvant. Immunization is repeated every two to three weeks for six cycles.

Isolation of B cells Producing Agonist Antibody from XenoMice (Abgenix, Fremont, Calif.)

When immunoreactive sera are detected in these animals, they are sacrificed and B cells collected for expansion into Abgenix's proprietary SLAM media, which allows for totally bypassing the hybridoma generation step. Positive pools of antibodies are screened for agonist activity against the cloned human receptor.

ELISA Positive B Cell Populations

B cells collected from the sacrificed mice are grown in 96-well plates and secrete intact antibody into the supernatant. Supernatants are collected and screened individually in ELISA binding assays to test for antibodies that bind the peptide immunogens used to immunize the mice. ELISA screens utilizing coupled immunogen peptides immobilized on high binding ELISA plates (ReactiBind Neutravidin EIA plates, Pierce Chemical Company, Rockford, Ill., Catalog No. 15507) are employed to detect peptide-binding antibodies secreted from the B cells. ELISA plates are coated with the biotinylated version of the immunogen-peptides at approximately 10 ng per well. Unbound peptide is washed with three well volumes of Dulbecco's phosphate buffered saline (D-PBS) (Invitrogen Corporation, Carlsbad, Calif., Catalog No. 14040-117) followed by incubation of B cell supernatant from cultured cells derived from immunized mouse spleens. After a suitable incubation time of 4-18 hours, ELISA plates are washed three times in D-PBS again and incubated with an anti-mouse rabbit horseradish peroxidase conjugated polyclonal antibody (Pierce Chemical Company, Rockford, Ill., Catalog No. 31444) for 2 hours. The plates are washed three times with D-PBS and developed using Kierkegaard and Perry's (Gaithersburg, Md.) peroxidase kit (Catalog No. 50-76-00) following manufacturer's instructions. Plates are read on a SpectraMax Model 340 (Molecular Devices, Sunnyvale, Calif.) Microplate wells that have A₄₅₀ readings of greater than 0.5 absorbance units at wavelength of 450 nm can be selected for cAMP functional assays described below. This ELISA screen is employed to identify which B cell isolates produce immunoreactive antibodies against the antigen peptides. Having identified the reactive clones, these supernatants can then be subjected to functional activity characterization.

Fluorescence-Activated Cell Sorting Assay for Detection of Candidate Antibody Binding to Live Transfected Cells Expressing human GLP1R

Materials

293G cells: Abbreviation for HEK 293G. A subcultivar of human embryonic kidney cell line derived from normal HEK 293 cells. Cells are routinely cultured in low calcium low serum medium LCSM (Hybridoma-SFM Gibco Cat. 12045-076, 1% FBS, Sigma F2442, Geneticin, 50 μg/ml Gibco, 10 mM HEPES, Gibco, at 37° C. and 90% humidity).

Secondary polyclonal FITC (fluorescein isothiocyanate) conjugated AffinityPure F(ab′) 2 goat anti-mouse IgG, Fcγ specific are obtained from Jackson ImmunoResearch Laboratories, WestGrove, Pa. (1:200 dilution in FACS buffer).

FACS Analysis

Cells were dispensed into the 96-well v-bottom polypropylene plates (Corning Costar 3363) at 5×10⁵/100ul/well. Plates are centrifuged at 1500 rpm for 5 minutes at 4° C. to pellet the cells. Testing hybridoma supernatants were diluted (in the range of 1:1˜1:9, usually 1:3) with a FACS buffer (0.1% BSA in PBS) into 96-well cell culture cluster (Corning Costar 3599). Hybridoma supernatants or the control antibody were diluted at the varying concentrations to suspend the cells. Cells were incubated at 4° C. for 1 hour. The cells were washed (suspension/centrifuging) three times with cold FACS buffer. Cells were resuspended in 100 μl of FITC conjugated AffinityPure F(ab′)2 fragment of goat anti-mouse IgG, Fcy specific antibodies (Jackson ImmunoResearch Lab, 1:200 dilution in FACS buffer) and incubated at 4° C. for 1 hour. Cells were then washed three times with cold FACS buffer and resuspended in 250 μl FACS buffer followed by transfer into cluster tubes (Corning Costar, 96-well polypropylene tube, Corning 4410). The cells were then analyzed on a FACS (BD Bioscience, San Jose, Calif.).

Functional Agonist Assays for Detection of Agonist Antibodies

The human GLP-1 receptor induces the production of intracellular cAMP upon activation by an agonist. The native ligand GLP-1 peptide as well as analog agonists like Exendin-4 are effective in stimulating this intracellular response. Antibodies that are agonists of this receptor will likewise induce cAMP production. One assay, the cyclase assay, is described in detail as follows:

Cyclase Assay for GLP-1 Receptor

GLP-1-stimulated cAMP production is determined using the FlashPlate Adenyl Cyclase Activation Assay (Catalog No. SMP004B, PerkinElmer Life and Analytical Sciences, Boston, Mass.). For evaluation of GLP-1 dose-response curve, GLP-1 is purchased from Sigma Chemical (St. Louis, Mo., Catalog No. G-9416). Forty μl of D-PBS (Invitrogen Carlsbad, Calif.,) are added to the wells, followed by the addition of 50 μl of CHO cells that express the human GLP-1 receptor (100,000 cells/well). Then 10 μL of GLP-1 (final concentration between 1×10⁻⁶ to 1×10⁻¹² M), prepared in stimulation buffer with 3-isobutyl-1-methylxanthine (IBMX) are added to each dose response control well and further incubated for 30 minutes at room temperature. Test wells used for evaluating agonist antibody supernatants receive B cell supernatant instead of the GLP-1 peptide. Afterward, 100 μl of the detection buffer containing radioactive tracer [¹²⁵I]succinyl cAMP tyrosine methyl ester are added, and the mixture is incubated at room temperature for an additional 90 minutes. The contents are then aspirated and the amount of [¹²⁵.]CAMP bound to the plate is measured by a TopCount microplate scintillation counter (Model No. C991200, PerkinElmer Life and Analytical Sciences, Boston, Mass.). Quantity of cAMP is calculated from a cAMP standard curve. Increasing concentrations of GLP-1 proportionally increases the cAMP concentration in the well. Typical EC50's derived for GLP-1 are approximately 20-50 picomolar.

Cis Bio HTRF Functional Assay

An alternative product to that recited in the above mentioned examples is Cisbio's (cyclic AMP dynamic 2 HTRF kit Cisbio Bedford, Mass.) homogenous time-resolved fluorescence (HTRF) kit for cyclic AMP. Detailed protocols are provided according to manufacturer's instructions. A brief review of this method is as follows:

The cells were dissociated from the flask using cell dissociation buffer (Invitrogen Catalog No. 13150-016) after reaching 80-90% confluency. A low speed spin for 5 minutes was used to pellet the cells, which were then resuspended in media to a concentration of 200,000 cells/ml. 100 μl of the cell suspension is added to each well (20,000 cells/well) of a 96 well plate by hand or using a MultiDrop. A 96 well Poly-D-Lysine plate was used for HEK cells and regular 96 well tissue culture treated plate was used for CHO cells. After plating, the cell plates were incubated at 37° C. and 5% CO₂ overnight.

Agonist-Specific vs. Allosteric Antibody Screening

As discussed above, functional assays designed to identify antibodies with pure agonist activity are performed without GLP-1 peptide agonist. Antibodies or derivative hybridomas are assayed with reporter cells incubated as described below and quantitated as stated. Assays designed to detect allosteric antibodies are performed in the presence of GLP-1 at concentrations ranging from 10-30 picomolar incretin.

On the day of the experiment, either candidate agonist or allosteric modulator hybridoma supernanatant, purified monoclonal antibody and additional peptide agonists (e.g., GLP-1) or antagonists (e.g., Exendin 9-39 antagonist) were diluted to the appropriate concentration using cAMP assay buffer in a 96 well plate. The volume in the agonist or allosteric antibody plate was usually 100 μl. The media was aspirated from the cell plate and the combined antibody plus peptide mixtures were transferred to cell plate by hand or using the BioMek FX. The cell plate was incubated at room temperature for 30 min. After the incubation the supernatant is aspirated from the cell plate and discarded. 50 μl of lysis buffer was added to each well and the plate was allowed to incubate for 60 minutes. 20 μl of the lysis supernatant from the cell plate was transferred by hand or using the Biomek FX to a 96 half-well black plate. Measurement of cAMP production was determined using the cAMP HTRF dynamic kit from CisBio. The protocol from the kit was followed as indicated except for a reduction in final cAMP assay volumes. The kit used 50 μL of cell supernatant with 25 μL of XL665 conjugate and 25 μL of cryptate conjugate. The protocol used 20 μL of cell supernatant with 10 μL of XL665 conjugate and 10 μL of cryptate conjugate. The assay plates were read on the BMG Rubystar fluorescent plate reader. A cAMP standard curve was used to determine molar concentrations of cAMP. Standard curve calculations were done in Excel using XLFit from IDBS. Graphing was done using GraphPad Prism software.

Antibodies that generate a statistically reproducible signal above basal cAMP production are identified and their corresponding source hybridomas are processed for expansion into larger tissue culture plates.

Pharmacology Models Utilized to Test Agonist Antibodies

Antibodies that are able to activate human GLP-1 receptor in CHO cells will be expanded for further studies. A subset of these antibodies may be able to trigger both the human and the murine forms of the GLP-1 receptor in in vitro assays. These antibodies can be utilized in animal models of obesity e.g., ob/ob or diet-induced obese (DIO) mice. These models will incorporate animals with a known genetic defect that confers a diabetic phenotype to the organism. This includes glucose intolerance, insulin resistance, dyslipidemia. Alternatively, rodents can be induced to become obese through a high fat, high calorie diet. Such diet-induced obesity models allow for the study of pharmacologic agents on a simulated rodent analog of Metabolic Syndrome X. GLP-1 receptor effects on satiety can be tested in such a model for ability to reduce excess body mass, fasting plasma glucose, dyslipidemia, percent glycated hemoglobin and HbA1_{c}.

### EXAMPLE 2

### Phage Display/Selection by Internalization or Binding/In Vitro Screening

Phage display technology (Sidhu (2000) Current Opinions in Biotechnology 11:610-616) is a very powerful technique developed to combine molecular diversity and reiterative selection strategies into one potent tool used in drug discovery. Phage antibody repertoires can be made larger and more diverse than is possible in an intact organism. In this technique, human antibodies are expressed as fusions with phage coat proteins of filamentous phage. Each infectious phage displays one or two antibody molecules on its surface. Entire libraries of these phage are then challenged with a “target” protein or hapten. Antibodies having high affinities with these targets bind and are retained during the washing phase of the selection protocol. High-affinity antibody displaying phage are then eluted off the target and amplified by growing in a host E. coli strain. Millions of new phage with the selected binding affinities are produced and submitted for another round of selection against the same target. Through reiterative selection only the antibodies that pass the selection criteria are retained, identified, and subcloned into an expression system for bulk production.

Synthetic humanized antibody libraries of immense diversity have been developed for phage display (Sidhu (2000) supra). These have yielded therapeutic antibodies that are currently approved drugs. One example of this application is D2E7 (Taylor (2001) Current Opinions in Rheumatology 13:164-169), a monoclonal antibody derived from a collaboration between Knoll Pharmaceuticals (now Abbott Bioresearch Center, Worcester, Mass.) and Cambridge Antibody Technology (CAT, Cambridge, England, UK). This antibody is postulated to prevent joint damage from autoimmune response. With synthetic libraries, antibodies that were previously impossible to generate, because they would result in either a fatal autoimmune response in laboratory animals or were clonally eliminated in early development, are now routinely generated with phage display techniques.

Since the inception of this technology, major refinements have been introduced that make the selection even more powerful. Many first generation libraries incorporated naïve (non-immunized) human antibody repertoires into the phage constructs. This was later improved by pre-immunizing mice with the antigen, then cloning the immunized murine repertoire into the phage library, enhancing success with a selected focused library of antibodies. Second generation libraries incorporated Cre-Lox recombination sites in the CDRs of the antibodies, further increasing the diversity of these libraries. Numerous refinements in the selection strategies have developed including coupling of receptor binding and internalization with phage reiterative selection (Heitner et al. (2001) Journal of Immunological Methods 248:17-30). This last method selects for phage that are co-internalized with over-expressed receptors, a protocol that may have immense selective pressure for agonistic antibodies that internalize with the receptor after triggering an activation event.

GLP-1 Receptor Phage Display Strategy

A large library of phage human antibodies are exposed to mammalian cells displaying the GLP-1 receptor. Lysates of untransfected cells are used to coat the unbound surfaces of the plating culture dish to reduce the probability that phage will bind to these sites. Phage that trigger an agonist response are internalized with the receptor. Phage that are inert are washed away with buffer. Cells with internalized phage rupture to liberate the agonistic phage antibodies. These phage are then selected and amplified for successive rounds of selection.

Antibodies from these phage are isolated using the polymerase chain reaction and cloned into expression vectors to express small quantities of monoclonal antibodies for in vitro analysis. Antibodies displaying GLP-1 receptor agonistic activity in cyclase assays are expanded for animal studies.

### EXAMPLE 3

### Converting an Inert Antibody to an Agonist Antibody by Conjugation

It is possible to convert an inert antibody into an agonist antibody by recombinant or chemical conjugation. An inert antibody is one that does not demonstrate agonist or antagonist activity against the human GLP-1 receptor. By using commonly available covalent coupling agents, it is possible to convert an inert antibody to an agonist antibody by conjugating agonist peptides to the antibody. This conjugation is effected in a manner that does not inactivate the agonist peptide's activity and yet allows the peptide to escape renal clearance. The agonist peptides can display synthetically modified non-naturally occurring amino acids that make them resistant to DPP IV degradation. A suitable chemical linker can be employed to couple the peptide to the antibody by attachment to functional groups on the surface of the antibody.

### EXAMPLE 4

### Development and Characterization of Allosteric mabs 5A10 and 9A10

The experiment was directed to immunizing wild-type and transgenic animals with a variety of immunogens including peptides, soluble receptor domains, in vivo expression plasmids, and intact mammalian cells expressing the human GLP1R.

Animals displaying seroconversion in ELISA and FACS assays were sacrificed and splenectomized, to derive cells for hybridoma fusions. Supernatants positive for binding to GLP1R were screened for agonist and allosteric modulatory activity against CHO K1 cells expressing the human GLP1R, using cAMP functional assays. Confirmed allosteric antibody hybridomas were expanded for medium scale production to support preclinical evaluation of candidate mAbs.

In particular, three monoclonal antibodies specific for GLP1R receptor were employed in a study [FIG. 5] using a fluorescent activated cell sorter (FACS) against three stably transfected cell lines (HEK 293G, K562, BaF 3) expressing the human GLP1 receptor. Cells of all three types (HEK 293G, K562, BaF 3) transfected with parent vector only were used as negative controls. Antibodies displayed no detectable binding to negative control cells which did not express human GLP1R. In contrast, the same antibodies elicited a 5- to 200-fold shift in fluorescent signal when incubated with transfected cells expressing approximately 100,000 receptors per cell, indicating positive robust binding to native GLP1R.

Monoclonal antibodies 5A10 and 9A10 were generated by immunizing mice with HEK 293G cells while 2A9 was developed using BaF3 cells as whole cell immunogens. None of the monoclonal antibodies displayed nonspecific binding to the parental form of their respective immunogen cell types, suggesting that their paratope specificity selectively targets an epitope on the GLP-1 receptor. (The hybridoma which produces monoclonal antibody 5A10 was deposited with the American Type Culture Collection, 10801 University Boulevard, Manassas, Va. 20110 on Jan. 16, 2006 under the terms of the Budapest Treaty and received designation ______, and the hybridoma which produces monoclonal antibody 9A10 was deposited with the American Type Culture Collection, 10801 University Boulevard, Manassas, Va. 20110 on Jan. 16, 2006 under the terms of the Budapest Treaty and received designation ______.)

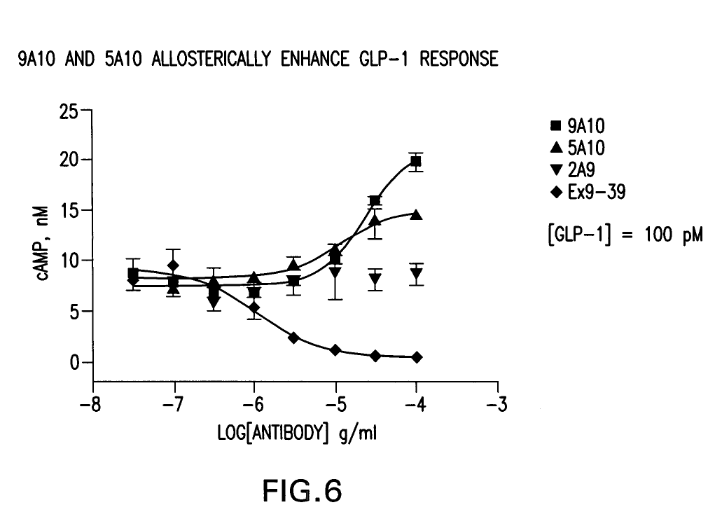

A modified screening protocol using submaximally stimulated cells was employed to identify allosterically active mAbs from all FACS positive candidates [FIG. 6]. The GLP-1 receptor is a Gs coupled GPCR that induces increased intracellular cAMP accumulation upon activation. GLP-1 peptide was introduced at 100 picomolar to half-stimulate the receptor and yet provide a sufficient window to detect enhanced activation thus allowing for identification of allosterically active mAbs.

Two allosteric mAbs of differing isotypes, 9A10 and 5A10 were identified using this technique. Both were developed from immunizations employing HEK 293G immunogen cells expressing human GLP1R. Both mAbs interact with an extracellular domain of the receptor, magnifying the cAMP physiological response to GLP-1. Monoclonal antibody 2A9, which demonstrated equivalent FACS binding properties to 9A10 and 5A10, failed to show allosteric modulatory action suggesting that mAb binding alone to the receptor is insufficient to elicit functional activity. Exendin 9-39 is an orthosteric competitive antagonist of the GLP1R employed as a control. The majority of mAbs screened by functional screening showed no allosteric activity against the receptor, suggesting the positive allosteric effect observed for 9A10 and 5A10 is not an artifact of high antibody concentration or reagent diluent on the intracellular cAMP read-out. This further underscores the structural requirement needed by allosteric antibodies against the GLP-1 receptor suggesting that simple binding is insufficient to achieve receptor modulation as was proposed for dimerizing antibodies to growth hormone-like hematopoietic receptors.

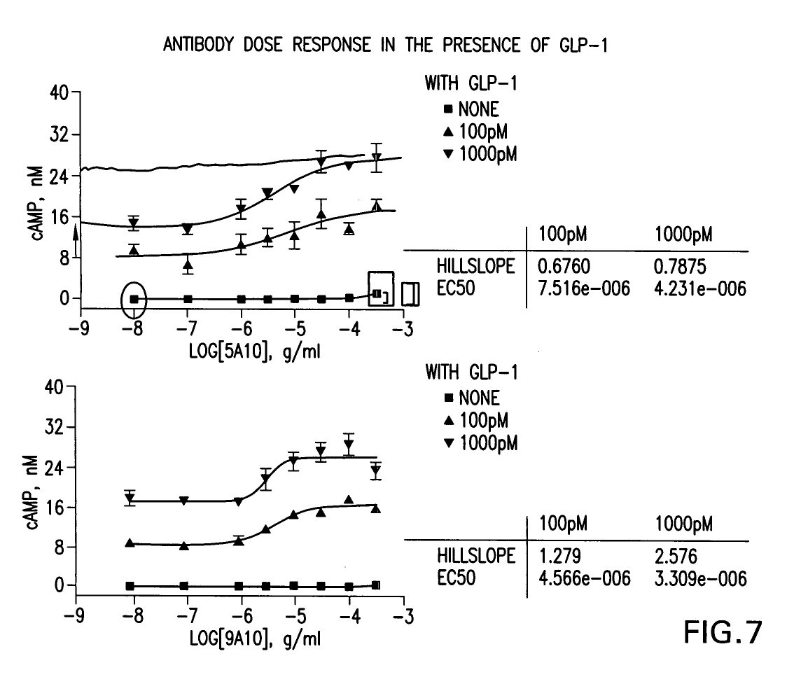

Both allosteric mAbs 5A10 and 9A10 were titrated against CHO K1 cells expressing the human GLP-1 receptor at two concentrations of GLP-1 ligand [FIG. 7]. At 100 picomolar and 1000 picomolar GLP-1, both allosteric mAbs induced a dosedependent increase in cAMP in response to the cognate ligand. This represented a 75-100% increase in the magnitude of the maximal physiological response as measured by cAMP levels.

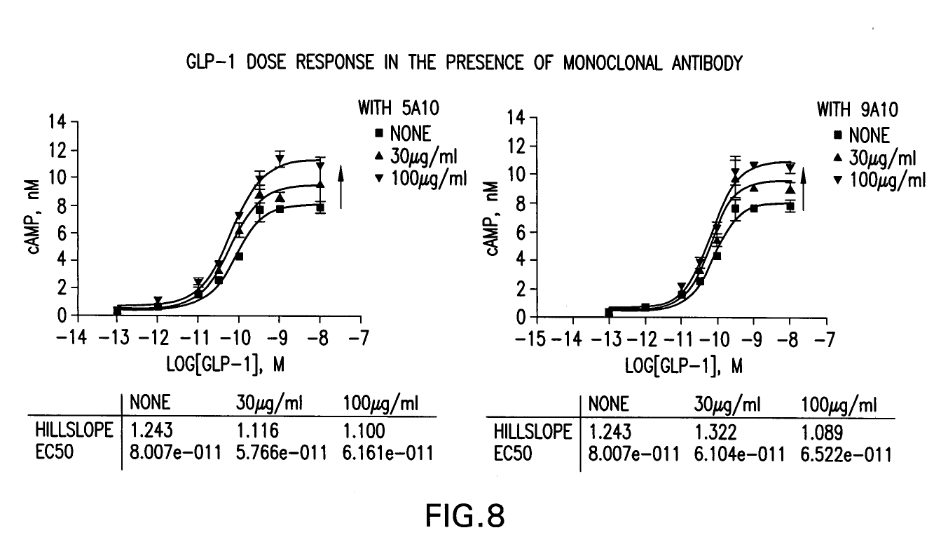

For each serial dose concentration of GLP-1 in the inflection range [FIG. 8], the allosteric mAbs induced an increase in the magnitude of cAMP response, suggesting the same level of physiological activation was achieved at a lower concentration of native ligand. This feature sensitizes the receptor to endogenous GLP-1 within the physiological concentration of incretin at 10-60 picomolar. Human plasma concentration of endogenous GLP-1 can range from 18-45 picomolar depending on the prandial state and meal size (Vilsbol et al. (2003) supra). Supra-physiological concentrations of incretin above this level have been exogenously introduced into subjects with marked improvement in therapeutic impact. Patient plasma glucose decreased by 5.5 mmol/L, HbAlc decreased by 1.3% and fructosamine levels were normalized. Fasting and 8 hr. mean concentrations of free fatty acids decreased by 30% and 23%. Gastric emptying was inhibited, resulting in average bodyweight reduction of 1.9 kg per subject, accompanied by a marked reduction in appetite. Both insulin sensitivity and beta-cell function improved (Zander et al. (2002) Lancet 359:824-830).

Analogous results are observed in patients receiving DPP IV inhibitors where a two-fold increase in circulating active GLP-1 results in dramatic improvements in glucose control, HbA1c and glucagon suppression (Ahren et al. (2004) supra). It is postulated from these findings that minute increases in GLP-1 activation can translate into therapeutically significant disease-modifying improvements.

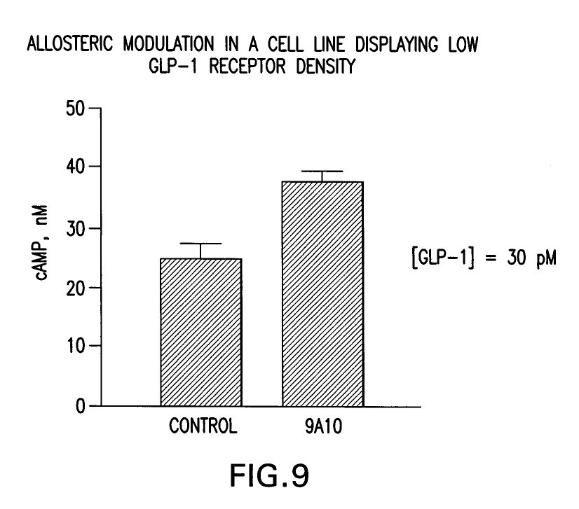

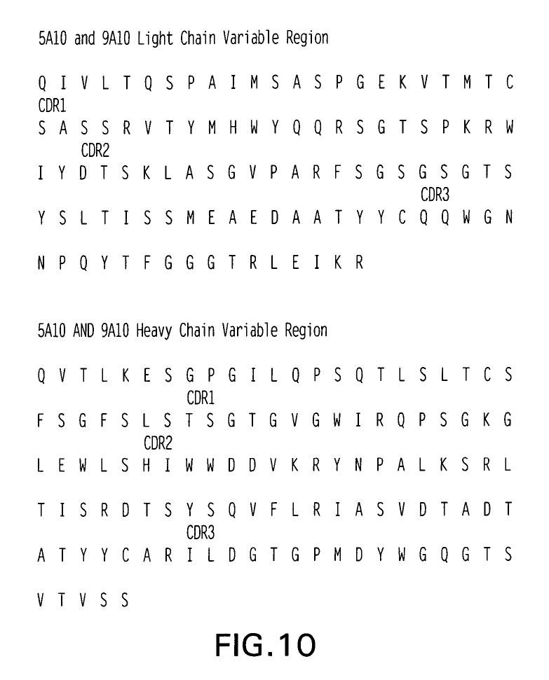

Allosteric antibodies were tested in cell based assays using low receptor copy CHO (DHFR⁻) cells [FIG. 9] to demonstrate antibody performance at lower receptor densities simulating natural islet receptor expression. Cells were stimulated with physiological concentration of GLP-1 similar to plasma levels expected in postprandial circulation. Antibody 9A10 induces allosteric enhancement of intracellular cAMP in response to GLP-1 even in cells expressing low copy numbers of receptor (4×10³). Antibodies are capable of exerting allosteric modulation on the GLP1R under these conditions suggesting that this class of mabs is potentially capable of therapeutic efficacy.

### EXAMPLE 5

### Sequencing of Monoclonal Antibodies

RNA was made from cell pellets containing 1×10⁸ hybridoma cells using the RNeasy Mini Kit (Qiagen Catalog No. 74104). RT-PCR was performed using the One-Step RT-PCR kit (Qiagen Catalog No. 210212) according to manufacturer's protocol. Mouse IgG primer set (Novagen Catalog No. 69831-3) was used for the PCR. PCR products were run on an agarose gel. Positive bands resulting from primer combinations MuIgVH5′D+MuIgGVH3′-2 for the heavy chains and from primer combinations MuIgKVL5′G+MuIgKVL3′-1 for the light chains were then cloned into the PCR 2.1 Topo vector (Invitrogen Catalog No. 46-0801) using the TOPO TA cloning kit (Invitrogen Catalog No. 45-0641). Colonies were screened for the insert using PCR. Four products from each heavy and light chain were submitted for sequencing. Four consensus sequences were used to determine the sequence for the VH region, and four consensus sequences were used for the determination of the VL region. The sequence was then imported into IgBlast for CDR determination.

1. A monoclonal antibody that binds to at least one epitope of a glucagon-like peptide-1 (GLP-1) receptor.

2. The monoclonal antibody of claim 1 wherein said epitope is located on one or more sites of said receptor selected from the group consisting of: a) the amino-terminal domain, b) extracellular loop 1, c) extracellular loop 2, d) extracellular loop 3, e) the intracellular carboxy-terminal domain, f) one or more of the three intracellular loops, g) a transmembrane peptide that transitions from the extracellular space to the transmembrane domain and h) a peptide that elicits activation of said receptor.

3. The monoclonal antibody of claim 2 wherein said antibody activates said receptor.

4. The monoclonal antibody of claim 3 wherein said activation results in a property selected from the group consisting of intracellular accumulation of cyclic AMP, calcium release and kinase mediated phosphorylation of a target protein substrate.

5. The monoclonal antibody of claim 1 wherein said antibody is humanized or fully human.

6. The monoclonal antibody of claim 1 wherein said antibody has been modified into an antibody-like entity selected from the group consisting of a scFv, a minibody, a dual-specific antibody and a single-domain antibody.

7. The monoclonal antibody of claim 1 wherein said antibody functions in a manner equivalent to the native ligand of said GLP-1 receptor or said antibody potentiates activity of said GLP-1 receptor bound to a different antibody.

8. A hybridoma which produces a monoclonal antibody, wherein said monoclonal antibody binds to the GLP-1 receptor.

9. The hybridoma of claim 8, wherein said monoclonal antibody activates said receptor.

10. A monoclonal antibody produced by said hybridoma of claim 8.

11. A method of treating a patient with Type 1 or Type 2 diabetes mellitus comprising administering to said patient a monoclonal antibody which binds to a GLP-1 receptor and activates or modulates said receptor, in an amount sufficient to effect said treatment.

12. The method of claim 11 wherein said monoclonal antibody causes at least one result selected from the group consisting of induction of insulin secretion, suppression of glucagon release, improvement of glycemic control, promotion of islet neogenesis, delay of gastric emptying and potentiation of glucose resistant islets.

13. A method of treating a patient with a neurodegenerative disorder, a cognitive disorder, memory disorder or learning disorder comprising administering to said patient a monoclonal antibody which binds to a GLP-1 receptor and activates or modulates said receptor, in an amount sufficient to effect said treatment.

14. The method of claim 13 wherein said neurodegenerative disorder is selected from the group consisting of dementia, senile dementia, mild cognitive impairment, Alzheimer-related dementia, Huntington's chores, tardive dyskinesia, hyperkinesias, mania, Morbus Parkinson, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis, glaucoma and Alzheimer's disease.

15. A method of treating a patient with a metabolic disorder selected from the group consisting of obesity, metabolic syndrome X and pathologies emanating from islet insufficiency comprising administering to said patient a monoclonal antibody which binds to a GLP-1 receptor and activates said receptor, in an amount sufficient to effect said treatment.

16. A method of producing a monoclonal antibody which binds to and activates or modulates a GLP-1 receptor comprising the steps of:
a) providing an antigen that comprises a structural feature of at least one receptor domain of said GLP-1 receptor;
b) exposing an antibody library or antibody fragment library to said antigen; and
c) selecting, from said antibody library or said antibody fragment library, an antibody that binds to said receptor and activates or modulates said receptor, wherein said antibody is a monoclonal antibody.

17. The method of claim 16 wherein said antigen is selected from the group consisting of: a) a cyclic peptide which mimics a structural loop of said GLP-1 receptor, b) a hybrid peptide comprising: 1) alternating regions of said GLP-1 receptor and 2) a non-GLP-1 receptor peptide that presents one or more loops and at least one extracellular domain, of said GLP-1 receptor, in an antigenic manner, and c) a hybrid peptide in which one peptide has been introduced into a full-length protein, wherein said one peptide and full-length peptide are functional regions of two different, structurally related proteins.

18. A method of producing a monoclonal antibody which binds to and activates or modulates a GLP-1 receptor comprising the steps of:
a) transfecting a nucleotide sequence encoding said GLP-1 receptor or a hybrid thereof into a cell line for a time and under conditions sufficient for said transfected cell line to express said GLP-1 receptor;
b) injecting said cell line into a mammal; and
c) generating a hybridoma from lymphocytes of said injected mammal of step b), wherein said hybridoma produces said monoclonal antibody which binds to and activates or modulates said GLP-1 receptor.

19. A method of producing a monoclonal antibody which binds to and activates or modulates the GLP-1 receptor comprising the steps of:
a) cloning a nucleic acid molecule encoding a GLP-1 receptor or hybrid thereof into a vector; and
b) screening an antibody library produced by said immunized mammal for an antibody produced against said antigen, wherein said monoclonal antibody binds to and activates or modulates said GLP-1 receptor and is monoclonal.

20. A method of producing a monoclonal antibody which binds to and activates or modulates the GLP-1 receptor comprising the steps of:
a) injecting a non-human mammal with a GLP-1 receptor antigen which stimulates an antibody response to said antigen in said mammal;
b) preparing and screening a phage display antibody library using immunoglobulin sequences from lymphocytes of said injected non-human mammal stimulated in vivo by exposure to said GLP-1 receptor antigen; and
a) selecting an antibody from said screened phage display antibody library, wherein said selected antibody binds to and activates or modulates said GLP-1 receptor antigen and is monoclonal.

21. A chemically modified antibody comprising at least one synthetic agonist peptide coupled to said monoclonal antibody of claim 1.

22. A pharmaceutical composition comprising: a) a monoclonal antibody that binds to and activates the GLP-1 receptor and b) a pharmaceutically acceptable carrier.

23. A monoclonal antibody which binds to the GLP-1 receptor and potentiates its function in the presence of an endogenous or exogenous agonist.

24. A chemically modified antibody comprising at least one synthetic agonist peptide coupled to said monoclonal antibody of claim 23.

25. A pharmaceutical composition comprising: a) a monoclonal antibody that binds to and modulates the GLP-1 receptor and b) a pharmaceutically acceptable carrier.

26. A pharmaceutical composition comprising:
a) a monoclonal antibody that 1) binds to and 2) activates or potentiates the GLP-1 receptor;
b) a pharmaceutical dose of a compound selected from the group consisting of a DPP IV inhibitor, a NEP inhibitor, a GLP-1 secretagogue, a GIP secretagogue and a protease resistant agonist of GLP1R; and
c) a pharmaceutically acceptable carrier.

27. A monoclonal antibody having at least one characteristic selected from the group consisting of:
a) improving sensitivity of the GLP-1 receptor such that physiological activation of said receptor is achieved in the presence of an endogenous or exogenous agonist;
b) magnifying response of said receptor such that resultant in vitro intracellular cyclic AMP response to said agonist is increased as compared to response with agonist alone; and
c) improving an in vivo response selected from the group consisting of: 1) glycemic control, 2) reduction of production of glycated hemoglobin, 3) reduction of glucagon secretion, 4) glucose sensitivity and 5) preservation of islet structure and function.

28. A monoclonal antibody (5A10) produced by a hybridoma designated by American Type Culture Collection patent deposit designation PTA-7322.

29. The hybridoma which produces said monoclonal antibody of claim 28.

30-31. (canceled)


SEQUENCE LISTING (11 sequences)

SEQ ID NO: 1 - DNA, 3071 bp, Homo sapiens
atggccggcgcccccggcccgctgcgccttgcgctgctgctgctcgggatggtgggcagggccggcccccgcccccagggtgccactgtgtccctctgggagacggtgcagaaatggcgagaataccgacgccagtgccagcgctccctgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccggttctgcacagctgaaggcctctggctgcagaaggacaactccagcctgccctggagggacttgtcggagtgcgaggagtccaagcgaggggagagaagctccccggaggagcagctcctgttcctctacatcatctacacggtgggctacgcactctccttctctgctctggttatcgcctctgcgatcctcctcggcttcagacacctgcactgcaccaggaactacatccacctgaacctgtttgcatccttcatcctgcgagcattgtccgtcttcatcaaggacgcagccctgaagtggatgtatagcacagccgcccagcagcaccagtgggatgggctcctctcctaccaggactctctgagctgccgcctggtgtttctgctcatgcagtactgtgtggcggccaattactactggctcttggtggagggcgtgtacctgtacacactgctggccttctcggtcttctctgagcaatggatcttcaggctctacgtgagcataggctggggtgttcccctgctgtttgttgtcccctggggcattgtcaagtacctctatgaggacgagggctgctggaccaggaactccaacatgaactactggctcattatccggctgcccattctctttggcattggggtgaacttcctcatctttgttcgggtcatctgcatcgtggtatccaaactgaaggccaatctcatgtgcaagacagacatcaaatgcagacttgccaagtccacgctgacactcatccccctgctggggactcatgaggtcatctttgcctttgtgatggacgagcacgcccgggggaccctgcgcttcatcaagctgtttacagagctctccttcacctccttccaggggctgatggtggccatcttatactgctttgtcaacaatgaggtccagctggaatttcggaagagctgggagcgctggcggcttgagcacttgcacatccagagggacagcagcatgaagcccctcaagtgtcccaccagcagcctgagcagtggagccacggcgggcagcagcatgtacacagccacttgccaggcctcctgcagctgagactccagcgcctgccctccctggggtccttgctgcaggccgggtggccaatccaggtgggagagacactcccagggacaagggaaggaagggacacacacacacacacacacacacacacacacacatacatcctgctttccctccccaaacccatcagacaggtaaatgggcagtgcctcctgggaccatggacacattttctcctaggagaagcagcctcctaatttgatcacagtggcgagaggagaggaaaaacgatcgctgtgaaaatgaggaggattgcttcttgtgaaaccacaggcccttggggttcccccagacagagccgcaaatcaaccccagactcaaactcaaggtcaacggcttattagtgaaactggggcttgcaagaggaggtggttctgaaagtggctcttctaacctcagccaaacacagagcgggagtgacgggagcctcctctgcttgcatcacttggggtcaccaccctcccctgtcttctctcaaagggaagctgtttgtgtgtctgggttgcttatttccctcatcttgccccctcatctcactgcccagtttctttttgaggggctttgtttgggccactgccagcagctgtttctggaaatggctgtaggtggtgttgagaaagaatgagcattgagacggtgctcgcttctcctccaggtatttgagttgttttggtgcctgcctctgccatgcccagagaatcagggcaggcttgccaccggggaacccagccctggggtatgagctgccaagtctattttaaagacgctcaagaatcctctggggttcatctagggacacgttaggaatgtccagactgtgggtgtagattacctgccacttccaggagcccagagggccaagagagacattgcctccacctctccttggaaatactttatctgtgaccacacgctgtctcttgagaatttggatacactctctagctttaggggaccatgaagagactctcttagggaaaccaatagtccccatcagcaccatggaggcaggctccccctgcctttgaaattcccccacttgggagcttgtatatacttcactcacttttctttattgctgtgaatagtctgtgtgcacaatgggcaattctgacttctcccatctagtggaaatgagcgaaatcatggttgtagtgatgttgtttgggagagtgcagtagtaattgatttgacccactcacacttggagctaattaaggtttgccctgcctgcagcctcccccacaaataatgaacagcagaaagactggacggggaaacctatcaatcctgcccccagccatggtgaggaagccccaagccatggtgacacacagcagcactgcagatagccagacacatggctatcctagagaggctggcaaggagttcgtggctgcaaaagaagtttctggagcaagagagagctcgctcttgggagtcaggacctccggggagagcagagggttccgacggattcctttatgagtcagtctctctctcccttttaaatggtgggaaccctccccaaaacctttccccagacacattctcctgtgcccctcagagaggcatgtgatgtgcaaggaaaataataggatgtaaaacacatcaagtagaaaatttcttatacttccaaaaaaaaaagc

SEQ ID NO: 2 - DNA, 3066 bp, Rattus norvegicus
tcctgagcgccccgccatggccgtcacccccagcctgctgcgcctggcgctcctgctgctcggggcggtgggcagggccggcccccgcccccagggtgccacggtgtccctctcagagacagtgcagaaatggagagagtatcggcaccagtgccaacgtttcctcacggaagcgccactcctggccacaggtctcttctgcaaccgaacctttgatgactacgcctgctggccagatgggcccccaggttcctttgtgaatgtcagttgcccctggtacctgccgtgggccagtagtgtgctccaagggcatgtgtaccggttctgcacggccgagggtatctggctgcataaggacaactccagcctgccctggagggacctgtcggagtgcgaagagtccaagcaaggagagagaaactcccctgaggaacagctcctgtcgctgtacattatctacacggtggggtacgcactttctttctctgccttggtcatcgcttcagccatccttgtcagcttcagacacttgcactgcaccaggaactacatccacctgaacctgtttgcgtccttcatcctccgagcactgtccgtcttcatcaaagacgctgccctcaagtggatgtatagcacggctgcgcaacagcaccagtgggatgggctcctctcgtatcaggactctctgggctgccgactggtgttcctgctcatgcaatactgcgtggcggccaactactactggttgctggtggaaggcgtgtatctgtacacactgctggccttctcggtgttctcggagcagcgcatcttcaagctgtacctgagcataggctggggagttccgctgctgttcgttatcccctggggcattgtcaagtatctctacgaggacgagggttgctggaccaggaactccaacatgaactattggctcatcatacgcttgcccattctctttgcaatcggggtcaacttccttgtcttcatccgggtcatctgcatcgtgatagccaagctgaaggctaatctcatgtgtaagaccgacatcaaatgcagactcgcgaagtccactctgacgctcatcccgcttctgggcacgcatgaagtcatctttgcctttgtgatggacgagcacgcccgaggaaccctacgcttcgtcaagctgttcacagagctctccttcacttccttccagggctttatggtggctgtcttgtactgctttgtcaacaatgaggtccagatggagtttcggaagagctgggagcgctggaggctggagcgcttgaacatccagagggacagcagcatgaaacccctcaagtgtcccaccagcagcgtcagcagtggggccacggtgggcagcagcgtgtatgcagccacctgccaaaattcctgcagctgagccccagtgctgcgcttcctgatggtccttgctgctggctgggtggccatcccaggtgggagagaccctggggacagggaatatgagggatacaggcacatgtgtgtgcgtgcccgcacaccacacacacacacacacacacacacacacacacacacacacacacacacacgctttcctcctcaaacctatcaaacaggcatcggcatcggcagtgcctcctgggaccacagacacatgttctccaaggagaacagcctgctaatttaatctcaggcgacaggaagagaggaagaaacaattgctgttaagacgaggaggacttcttcctgttaaagctgcaaggcccttggggttccctcggacagaactgcaaatcaaccccggaactctcgctcaagggcaattgctgacgggtggaacttgggcttgcgagaggaggcaggtccatgagagacctgcccttggaacctcagccagcacagcgctcatcaaggtgagctggctgtgctgtgtgcacggctggggttgtcacctacatcagccttcctctcggacaagaggcttttctctgtgcatctggagtgccgccattcctccatctgcccgttcatccgccatcctgtctttgccttggggagggggaggtttgttgaagtcatgccgtgcagctctttctggaaatatctgtggatggtgttgaagataagcatgggggagatacaacagaggcagtctttgcccatggccacttcttgcctggtcctttaagccactttgctgcttggtttctgccctgcatgggtactactagggcaggtcccaagttgagaagcccagaggtgaggtgtgaaccctcagttctgttgtaaagatgctcaaataccctctaaggttcatctaaaggagtaacctgcctaggggtgctgttgacctgaaatcaagaggaccaaaggatccattgccaacaccccccatcccccacacacacctcatctgtgaccagagtctatgctttgaatcagaatgggctatatcctctgacctcagaggctatgacccagaagagattcttccctgaatcctcccactttgcacacatatagactttatccttcttcactctgtgtctattcaaacgtataattctggtttctctcaccccacggaagaactagatcacagcaactgttatgtttgagggagtgggggagaaggtgattgatttgaccccctctcccccaccggtgttgataagtagcgtctgtcccacctccagactccacccacacataatgagcagcacatagaccaggatggggggggtggtatatcatgcttgccctcctccaaccactatgagaaggctagcagaagacaccactgcacagacccaagtccaaggactgcctcccagggaattaggcagtgacttcctagaggccaagaaagactccaagagctggagaagaatcctagtcgatctggatctcttttgaggttggggttggggtggctttcaatggattctctcatgaggcttatctctccctcatcccgtggagagtgggggaccctccctagtgctcacactagacactgtgccccttggagaggcataaggcatgtatgggagataataatgggctataaaacat

SEQ ID NO: 3 - DNA, 1502 bp, Mus musculus
atggccagcaccccaagcctcctgcgcctggcgctcctgctgctcggggcggtgggcagggccggcccccgccctcagggtaccacggtgtccctctcagagacggtgcagaaatggagagaataccggcggcagtgccagcatttcctcacggaagcgccactcctggccacaggcctcttctgcaaccggacctttgatgactatgcctgctggccggatgggcccccaggttccttcgtgaatgtcagctgcccctggtacctgccctgggccagtagtgtgctacaaggccatgtgtaccggttctgcacagctgagggtctctggctacataaggacaactccagcctgccctggagggacctgtcggagtgtgaagagtctaagcgaggggagagaaactttcctgaggaacagctcctgtccctgtacattatctacacagtggggtacgcactttctttctccgccttggtcatcgcttcagccatccttgttggcttcagacacttgcactgcaccaggaactacatccacctgaacctgtttgcatccttcatcctccgagcactgtccgtcttcatcaaagatgctgccctcaagtggatgtatagcacggctgcccagcaacaccagtgggatgggctcctctcctatcaggactctctaggctgccgactggtgttcctgctcatgcagtactgcgtggcagccaactactactggttgctggtggaaggcgtgtatctatacacactgctggccttctcggtgttctcggagcagcgcatcttcaagctgtatctgagcataggctggggagttccgctgctgttcgttatcccatgggggattgtcaagtatctctatgaggacgagggctgctggactaggaactccaatatgaactactggctcatcatccgattgcccattctctttgctatcggcgtcaactttcttatcttcatccgtgtcatctgcatagtggtctccaaactgaaggctaatctcatgtgcaagactgacatcaaatgcaggcttgccaagtccactctgactctcatcccccttctggggacacatgaagtcatctttgcctttgtgatggacgaacacgcccgaggaaccctacgcttcatcaagctgttcacagaactctccttcacttccttccagggcttgatggtggctatcctgtactgctttgtcaacaatgaggtccagatggagtttcggaaatgctgggagcgctggaggctggagcacttgaatatccagagagactgcagcatgaagcccctcaagtgtcccaccagcagcgtcagcagtggggccacagtggcagcagcgtgtatgcagccacctgccagagttcctacagctgagcccgcagtgccggtcctccctgttggtccttcgttctggctgggtggccatcccaggtgggagagatcctggggatagggaaggtgaaggacacaggcacaccggaattc

SEQ ID NO: 4 - protein, 463 aa, Homo sapiens
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYIIYTVGYALSFSALVIASAILLGFRHLHCTRNYIHLNLFASFILRALSVFIKDAALKWMYSTAAQQHQWDGLLSYQDSLSCRLVFLLMQYCVAANYYWLLVEGVYLYTLLAFSVFSEQWIFRLYVSIGWGVPLLFVVPWGIVKYLYEDEGCWTRNSNMNYWLIIRLPILFGIGVNFLIFVRVICIVVSKLKANLMCKTDIKCRLAKSTLTLIPLLGTHEVIFAFVMDEHARGTLRFIKLFTELSFTSFQGLMVAILYCFVNNEVQLEFRKSWERWRLEHLHIQRDSSMKPLKCPTSSLSSGATAGSSMYTATCQASCS

SEQ ID NO: 5 - protein, 463 aa, Rattus norvegicus
MAVTPSLLRLALLLLGAVGRAGPRPQGATVSLSETVQKWREYRHQCQRFLTEAPLLATGLFCNRTFDDYACWPDGPPGSFVNVSCPWYLPWASSVLQGHVYRFCTAEGIWLHKDNSSLPWRDLSECEESKQGERNSPEEQLLSLYIIYTVGYALSFSALVIASAILVSFRHLHCTRNYIHLNLFASFILRALSVFIKDAALKWMYSTAAQQHQWDGLLSYQDSLGCRLVFLLMQYCVAANYYWLLVEGVYLYTLLAFSVFSEQRIFKLYLSIGWGVPLLFVIPWGIVKYLYEDEGCWTRNSNMNYWLIIRLPILFAIGVNFLVFIRVICIVIAKLKANLMCKTDIKCRLAKSTLTLIPLLGTHEVIFAFVMDEHARGTLRFVKLFTELSFTSFQGFMVAVLYCFVNNEVQMEFRKSWERWRLERLNIQRDSSMKPLKCPTSSVSSGATVGSSVYAATCQNSCS

SEQ ID NO: 6 - protein, 489 aa, Mus musculus
MASTPSLLRLALLLLGAVGRAGPRPQGTTVSLSETVQKWREYRRQCQHFLTEAPLLATGLFCNRTFDDYACWPDGPPGSFVNVSCPWYLPWASSVLQGHVYRFCTAEGLWLHKDNSSLPWRDLSECEESKRGERNFPEEQLLSLYIIYTVGYALSFSALVIASAILVGFRHLHCTRNYIHLNLFASFILRALSVFIKDAALKWMYSTAAQQHQWDGLLSYQDSLGCRLVFLLMQYCVAANYYWLLVEGVYLYTLLAFSVFSEQRIFKLYLSIGWGVPLLFVIPWGIVKYLYEDEGCWTRNSNMNYWLIIRLPILFAIGVNFLIFIRVICIVVSKLKANLMCKTDIKCRLAKSTLTLIPLLGTHEVIFAFVMDEHARGTLRFIKLFTELSFTSFQGLMVAILYCFVNNEVQMEFRKCWERWRLEHLNIQRDCSMKPLKCPTSSVSSGATVAAACMQPPARVPTAEPAVPVLPVGPSFWLGGHPRWERSWG

SEQ ID NO: 7 - protein, 30 aa, Mus musculus
AALKWMYSTAAQQHQWDGLLSYQDSLSCRE

SEQ ID NO: 8 - protein, 16 aa, Mus musculus
CYEDEGCWTRNSNMNE

SEQ ID NO: 9 - protein, 18 aa, Mus musculus
CDEHARGTLRFIKLFTEE

SEQ ID NO: 10 - protein, 108 aa, Mus musculus
QIVLTQSPAIMSASPGEKVTMTCSASSRVTYMHWYQQRSGTSPKRWIYDTSKLASGVPARFSGSGSGTSYSLTISSMEAEDAATYYCQQWGNNPQYTFGGGTRLEIKR

SEQ ID NO: 11 - protein, 120 aa, Mus musculus
QVTLKESGPGILQPSQTLSLTCSFSGFSLSTSGTGVGWIRQPSGKGLEWLSHIWWDDVKRYNPALKSRLTISRDTSYSQVFLRIASVDTADTATYYCARILDGTGPMDYWGQGTSVTVSS